(19) United States
(12) Patent Application Publication
(10) Pub. No.: US 2013/0131057 A1
(43) Pub. Date: May 23, 2013

(54) NEW BICYCLIC COMPOUNDS AS PI3-K AND MTOR INHIBITORS

(71) Applicants: Joaquin Pastor Fernández, Madrid (ES); Sonia Martínez Gonzalez, Madrid (ES); Antonlo Rodriguez Hergueta, Madrid (ES); Francisco Javier Ramos Lima, Madrid (ES); Rosa Maria Alvarez Escobar, Madrid (ES); Ana Isabel Higueras Hernandez, Madrid (ES)

(72) Inventors: Joaquin Pastor Fernández, Madrid (ES); Sonia Martínez Gonzalez, Madrid (ES); Antonlo Rodriguez Hergueta, Madrid (ES); Francisco Javier Ramos Lima, Madrid (ES); Rosa Maria Alvarez Escobar, Madrid (ES); Ana Isabel Higueras Hernandez, Madrid (ES)

(73) Assignee: CENTRO NACIONAL DE INVESTIGACIONES ONCOLOGICAS (CNIO, Madrid (ES)

(21) Appl. No.: 13/697,499

(22) PCT Filed: May 13, 2011
(86) PCT No.: PCT/GB2011/000735

§ 371 Date: Jan. 28, 2013

(30) Foreign Application Priority Data
May 13, 2010 (EP) 10380070.2

(51) Int. Cl. C07D 487/04; A61K 45/06; A61K 31/5377

(52) U.S. Cl. CPC C07D 487/04; A61K 31/5377; A61K 45/06
USPC 514/233.2; 544/113; 544/117

(57) ABSTRACT

There is provided compounds of formula (I), wherein A₁, A₄, A₄ₐ, A₅, B¹, B^{1a}, B², B^{2a}, B³, B^{3a}, B⁴, B^{4a} and R³ have meanings given in the description, and pharmaceutically-acceptable esters, amides, solvates or salts thereof, which compounds are useful in the treatment of diseases in which inhibition of a protein or lipid kinase (e.g. a PI3-K and/or mTOR) is desired and/or required, and particularly in the treatment of cancer or a proliferative disease.

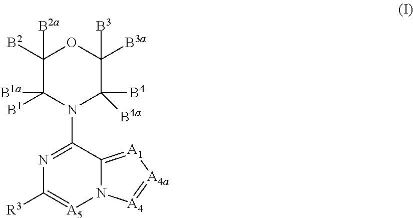

### FIELD OF THE INVENTION

This invention relates to novel pharmaceutically-useful compounds, which compounds are useful as inhibitors of protein or lipid kinases (such as inhibitors of the phosphoinositide 3′OH kinase (PI3 kinase) family, particularly the PI3K class I sub-type. The compounds may also be useful as inhibitors of the mammalian target of rapamycin (mTOR)). The compounds are of potential utility in the treatment of diseases such as cancer. The invention also relates to the use of such compounds as medicaments, to the use of such compounds for in vitro, in situ and in vivo diagnosis or treatment of mammalian cells (or associated pathological conditions), to pharmaceutical compositions containing them, and to synthetic routes for their production.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

Phosphatidylinositol 3-kinases (PI3Ks) are a family of lipid and serine/threonine kinases that catalyze the phosphorylation of the membrane lipid phosphatidylinositol (PI) on the 3′-OH of the inositol ring to produce phosphoinositol-3-phosphate (PIP), phosphoinositol-3,4-diphosphate (PIP₂) and phosphoinositol-3,4,5-triphosphate (PIP₃), which act as recruitment sites for various intracellular signalling proteins, which in turn form signalling complexes to relay extracellular signals to the cytoplasmic face of the plasma membrane. These 3′-phosphoinositide subtypes function as second messengers in intra-cellular signal transduction pathways (see e.g. Trends Biochem. Sci 22 87,267-72 (1997) by Vanhaesebroeck et al.; Chem. Rev. 101 (8), 2365-80 (2001) by Leslie et al (2001); Annu. Rev. Cell. Dev. Boil. 17, 615-75 (2001) by Katso et at; and Cell. Mol. Life. Sci. 59 (5), 761-79 (2002) by Toker et al).

Multiple PI3K isoforms categorized by their catalytic subunits, their regulation by corresponding regulatory subunits, expression patterns and signalling specific funtions (p110α, β, δ, γ) perform this enzymatic reaction (Exp. Cell. Res. 25 (1), 239-54 (1999) by Vanhaesebroeck and Katso et al., 2001, above).

The closely related isoforms p110α and β are ubiquitously expressed, while δ and γ are more specifically expressed in the haematopoietic cell system, smooth muscle cells, myocytes and endothelial cells (see e.g. Trends Biochem. Sci. 22 (7), 267-72 (1997) by Vanhaesebroeck et al). Their expression might also be regulated in an inducible manner depending on the cellular, tissue type and stimuli as well as disease context. Inductibility of protein expression includes synthesis of protein as well as protein stabilization that is in part regulated by association with regulatory subunits.

Eight mammalian PI3Ks have been identified so far, including four class I PI3Ks. Class Ia includes PI3Kα, PI3Kβ and PI3Kδ. All of the class Ia enzymes are heterodimeric complexes comprising a catalytic subunit (p110α, p110β or p110δ) associated with an SH2 domain containing p85 adapter subunit. Class Ia PI3Ks are activated through tyrosine kinase signalling and are involved in cell proliferation and survival. PI3Kα and PI3Kβ have also been implicated in tumorigenesis in a variety of human cancers. Thus, pharmacological inhibitors of PI3Kα and PI3Kβ are useful for treating various types of cancer.

PI3Kγ, the only member of the Class Ib PI3Ks, consists of a catalytic subunit p110γ, which is associated with a p110 regulatory subunit. PI3Kγ is regulated by G protein coupled receptors (GPCRs) via association with βγ subunits of heterotrimeric G proteins. PI3Kγ is expressed primarily in hematopoietic cells and cardiomyocytes and is involved in inflammation and mast cell function. Thus, pharmacological inhibitors of PI3Kγ are useful for treating a variety of inflammatory diseases, allergies and cardiovascular diseases.

These observations show that deregulation of phosphoinositol-3-kinase and the upstream and downstream components of this signalling pathway is one of the most common deregulations associated with human cancers and proliferative diseases (see e.g. Parsons et al., Nature 436:792 (2005); Hennessey et al., Nature Rev. Drug Discovery 4: 988-1004 (2005).

The mammalian target of rapamycin (mTOR) also known as FK506 binding protein 12-rapamycin associated protein 1 (FRAP1) is a protein which in humans is encoded by the FRAP1 gene. mTOR is a serine/threonine protein kinase that regulates cell growth, cell proliferation, cell motility, cell survival, protein synthesis, and transcription. The inhibition of mTORs are believed to be useful for treating various diseases/conditions, such as cancer (for example, as described in Easton et al. (2006). “mTOR and cancer therapy”. Oncogene 25 (48): 6436-46).

For the treatment of cancer, targeted therapies are becoming more important. That is, therapy that has the effect of interfering with specific target molecules that are linked to tumor growth and/or carcinogenesis. Such therapy may be more effective than current treatments (e.g. chemotherapy) and less harmful to normal cells (e.g. because chemotherapy has the potential to kill normal cells as well as cancerous cells). This, and also the fact that targeted therapies may be selective (i.e. it may inhibit a certain targeted molecule more selectively as compared to other molecular targets, e.g. as described hereinafter), may have the benefit of reducing side effects and may also have the benefit that certain specific cancers can be treated (also selectively). The latter may in turn also reduce side effects.

Hence, it is a clear goal of current oncologists to develop targeted therapies (e.g. ones that are selective). In this respect, it should be pointed out that several different molecular targets may exist that are linked to certain diseases (e.g. cancer). However, one simply cannot predict if a therapy (e.g. a small molecule as a therapeutic) that interferes with or inhibits one target molecule could inhibit a different molecular target (be it one that will ultimately have the effect of treating the same disease or a different one).

The listing or discussion of an apparently prior-published document in this specification should not necessarily be taken as an acknowledgement that the document is part of the state of the art or is common general knowledge.

US patent application US 2009/0163489 and international patent application WO 2009/085230 both disclose various molecules containing a 6,5-fused bicyclic core, which may be useful as inhibitors of PI3 kinase (PI3-K). However, these documents do not relate to 6,5-bicyclic compounds that are substituted on the 6-membered ring with at least two substituents, an aromatic group and a morpholinyl group.

International patent applications WO 2007/127175 and WO 2006/046040 both disclose various thienopyrimidines and furopyrimidines, of potential use as PI3-K inhibitors. However, these documents do not disclose or suggest any other 6,5-fused bicyclic compounds.

International patent application WO 2004/092177 discloses various triazolopyrazines for use in modulating the A₂ₐ adenosine receptor signalling pathways. International patent applications WO 2006/027346, WO 2007/032936, WO 2005/042537, WO 2007/088168, WO 2008/131050, WO 03/000693, WO 2004/005290 and WO 2004/005291 and US patent application US 2006/0084650 (and international patent application WO 2006/044687) disclose various bicyclic compounds that may be useful for treating diseases/disorders such as cancer, pain, neurodegenerative disorders and/or that may be useful as kinase inhibitors. However, these documents do not relate to such bicycles that are directly substituted with both an aromatic group and a morpholinyl group.

International patent applications WO 2008/113469 and WO 2009/007029 disclose various compounds including bicyclic compounds, for use in treating diseases such as haematological diseases. However, these documents do not relate to bicycles that are substituted with a morpholinyl group.

Journal article Chorvat et al., J. Med. Chem. 1999, 42, 833 discloses various bicyclic compounds that may possess biological activity. However, there is no disclosure of 6,5-fused bicycles in which the 6-membered ring is directly substituted with an aromatic group.

International patent application WO 2005/035532 discloses various triazolopyrazinones that may be useful in the treatment of asthma or another glycogen synthase kinase mediated condition. However, this document only discloses 6,5-bicyclic compounds in which there is a carbonyl group attached to the 5-membered ring.

US patent application (and equivalent application WO 2007/038314) as well as international patent application WO 2008/116064 both disclose various compounds, including bicycles, which may be useful in the treatment of inflammatory and immune diseases. However, these documents do not predominantly relate to 6,5-fused bicyclic compounds that are substituted with both an aromatic group and a morpholinyl group.

German patent application DE 2424334 and US patent U.S. Pat. No. 3,995,039 disclose various bicyclic compounds for use as medicaments (e.g. in the treatment of asthma and related diseases). However, these documents do not mention that the compounds disclosed therein may be useful as kinase inhibitors, and do not primarily relate to 6,5-fused bicycles that are substituted with both an aromatic group and a morpholinyl group.

International patent application WO 03/044021 discloses various bicycles for use as mediators of pro-inflammatory cytokines, which may therefore be useful in the treatment of e.g. pain. There is no specific disclosure in this document of 6,5-fused bicycles that are substituted on the 6-membered ring with morpholinyl and only one other aromatic group.

International patent application WO 2010/119264 discloses various imidazopyrazines for use as kinase inhibitors, which imidazopyrazines may be substituted with an aromatic group and a morpholinyl group. However, this document only relates to imidazopyrazines.

International patent application WO 2009/085230 and US patent application US 2009/085230 disclose various 6,5-fused bicyclic compounds. However, this application does not predominantly relate to bicycles in which the 6-membered ring is substituted with two certain substituents.

### DISCLOSURE OF THE INVENTION

According to the invention, there is now provided a compound of formula I,

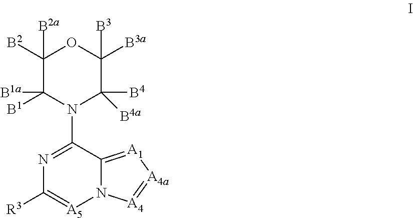

wherein:
A₁ represents N or C(R¹);
A₄ represents N or C(R^{1a}),
A₄ₐ represents N or C(R^{1b});
wherein at least one of A₄ and A₄ₐ does not represent N;
A₅ represents N or C(R²);
each B¹, B^{1a}, B², B^{2a}, B³, B^{3a}, B⁴ and B^{4a} independently represent hydrogen or a substituent selected from halo, —C(═Y)—R^{10a}, —C(═Y)OR^{10a}, —C(═Y)N(R^{10a})R^{11a},
—S(O)₂N(R^{10a})R^{11a}, C₁₋₁₂ alkyl, heterocycloalkyl (which latter two groups are optionally substituted by one or more substituents selected from ═O and E¹), aryl and/or heteroaryl (which latter two groups are optionally substituted by one or more substituents selected from E²); or
any two B¹, B^{1a}, B², B^{2a}, B³, B^{3a}, B⁴ and B^{4a} substituents that are attached to the same carbon atom (i.e. B¹ and B^{1a}; B² and B^{2a}; B³ and B^{3a}; and/or B⁴ and B^{4a}) may together form a ═O group;
or, any two B¹, B^{1a}, B², B^{2a}, B³, B^{3a}, B⁴ and B^{4a} substituents may be linked together to form a further 3- to 12-membered (e.g. 3- to 6-membered) ring, optionally containing (in addition to the atom(s) of the morpholine ring) one or more (e.g. two or, preferably, one) heteroatom(s) (preferably selected from sulfur, oxygen and nitrogen), which ring optionally contains one or more (e.g. one to three) double bonds, and which ring is itself optionally substituted by one or more substituents selected from halo, ═O and C₁₋₃ alkyl optionally substituted by one or more fluoro atoms;
R¹ and R² (when present) independently represent hydrogen or a substituent selected from halo, —CN, —OR^{10b}, —N(R^{10b})R^{11b}, —C(O)N(R^{10b})R^{11b}, C₁₋₁₂ (e.g. C₁₋₆) alkyl and heterocycloalkyl (e.g. a 3- to 7-membered heterocycloalkyl), which latter two groups are optionally substituted by one or more substituents selected from E³ and ═O;
one of R^{1a} and R^{1b} is present and represents —C(═Y)N(R^{10a})R^{11a} or —C(═Y)—R^{10a} and the other (when/if present) independently represents hydrogen or Q¹;
Q′ represents:
halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, alkyl, heterocycloalkyl (which latter two groups are optionally substituted by one or more substituents selected from ═O and E⁴), aryl or heteroaryl (which latter two groups are optionally substituted by one or more substituents selected from E^{4a});
R³ represents:
(i) a bicyclic aryl or heteroaryl group (both of which are optionally substituted by one or more substituents selected from E⁵); or
(ii) a fragment of formula IA or IB,

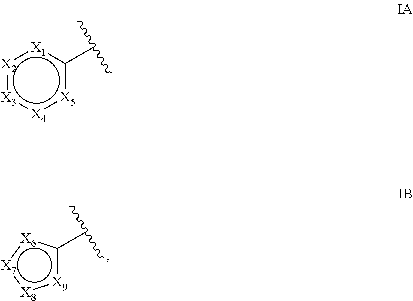

wherein
any one of X₁, X₂, X₃, X₄ and X₅ (e.g. X₃) represents C(R^{2a}), a second one of X₁, X₂, X₃, X₄ and X₅ represents C(R^{2b}), and the remaining three independently represent C(R^{2b}) or N; or
any one, two or three of X₁, X₂, X₃, X₄ and X₅ may represent N and those remaining represent C(H);
X₆, X₇, X₅ and X₉ independently represent C(R^{2b}), N, O or S;
each R^{2b} independently represents hydrogen, halo, —CN or R^{2a};
each R^{2a} independently represents, on each occasion when used herein, —N(R^{5a})R^{5b}, —N(R^{5c})—C(═Y)—R^{5d}, —N(R^{5e})—C(═Y)—N(R^{5f}), —N(R^{5g})—C(O)—OR^{5h}, —N(R⁵ⁱ)—OR^{5j}, —OR^{5k}, —N(R^{5m})—S(O)₂—R⁵ⁿ or C₁₋₁₂ (e.g. C₁₋₆) alkyl optionally substituted by one or more halo (e.g. fluoro) atoms;
each R^{5a}, R^{5b}, R^{5C}, R^{5d}, R^{5e}, R^{5f}, R^{5g}, R^{5h}, R⁵ⁱ, R^{5j}, R^{5k}, R^{5m} and R⁵ⁿ independently represent hydrogen, C₁₋₁₂ (e.g. C₁₋₆) alkyl (optionally substituted by one or more halo (e.g. fluoro) atoms), heterocycloalkyl, aryl or heteroaryl (which latter three groups are optionally substituted by one or more substituents selected from halo and C₁₋₄ alkyl);
each R^{10a}, R^{11a}, R^{10b}, R^{11b} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁶), aryl or heteroaryl (which latter two groups are optionally substituted by one or more substituents selected from E⁷); or
any relevant pair of R^{10a} and R^{11a} or R^{10b} and R^{11b} (for example, when attached to the same atom, adjacent atom (i.e. 1,2-relationship) or to atoms that are two atoms apart, i.e. in a 1,3-relationship) may be linked together to form (e.g. along with the requisite nitrogen atom to which they may be attached) a 4- to 20- (e.g. 4- to 12-) membered ring, optionally containing one or more heteroatoms (for example, in addition to those that may already be present, e.g. (a) heteroatom(s) selected from oxygen, nitrogen and sulfur), optionally containing one or more unsaturations (preferably, double bonds), and which ring is optionally substituted by one or more substituents selected from ═O, ═S, ═N(R²⁰) and E⁸;
each E¹, E², E³, E⁴, E^{4a}, E⁵, E⁶, E⁷ and E⁸ independently represents, on each occasion when used herein:

### (i) Q⁴;

(ii) C₁₋₁₂ alkyl optionally substituted by one or more substituents selected from ═O and Q⁵; or
any two E¹, E², E³, E^{4a}, E⁵, E⁶, E⁷ and E⁸ groups, for example on C₁₋₁₂ alkyl groups, e.g. when they are attached to the same or adjacent carbon atoms, or on adjacent atoms of an aryl group, may be linked together to form a 3- to 12-membered ring, optionally containing one or more (e.g. one to three) unsaturations (preferably, double bonds), and which ring is optionally substituted by one or more substituents selected from ═O and J¹;
each Q⁴ and Q⁵ independently represent, on each occasion when used herein: halo, —CN, —NO₂, —N(R²⁰)R²¹, —C(═Y)—R²⁰, —C(═Y)—OR²⁰, —C(═Y)N(R²⁰)R²¹, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;

each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or

any relevant pair of R²⁰, R²¹ and R²², may (for example, when attached to the same atom, adjacent atom (i.e. 1,2-relationship) or to atoms that are two atoms apart, i.e. in a 1,3-relationship) be linked together to form (e.g. along with the requisite nitrogen atom to which they may be attached) a 4- to 20- (e.g. 4- to 12-) membered ring, optionally containing one or more heteroatoms (for example, in addition to those that may already be present, e.g. (a) heteroatom(s) selected from oxygen, nitrogen and sulfur), optionally containing one or more unsaturations (preferably, double bonds), and which ring is optionally substituted by one or more substituents selected from J⁶ and ═O;

each J¹, J², J³, J⁴, J⁵ and J⁶ independently represents, on each occasion when used herein:

### (i) Q⁷;

(ii) C₁₋₆ alkyl or heterocycloalkyl, both of which are optionally substituted by one or more substituents selected from ═O and Q⁸;
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may (for example when attached to the same or adjacent atoms) be linked together to form, a 3- to 8-membered ring, optionally containing one or more heteroatoms (for example, in addition to those that may already be present, heteroatoms selected from oxygen, nitrogen and sulfur), optionally containing one or more unsaturations (preferably, double bonds), and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms;
or a pharmaceutically acceptable ester, amide, solvate or salt thereof.
which compounds, esters, amides, solvates and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

By “pharmaceutically acceptable ester, amide, solvate or salt thereof”, we include salts of pharmaceutically acceptable esters or amides, and solvates of pharmaceutically acceptable esters, amides or salts. For instance, pharmaceutically acceptable esters and amides such as those defined herein may be mentioned, as well as pharmaceutically acceptable solvates or salts.

Pharmaceutically acceptable esters and amides of the compounds of the invention are also included within the scope of the invention. Pharmaceutically acceptable esters and amides of compounds of the invention may be formed from corresponding compounds that have an appropriate group, for example an acid group, converted to the appropriate ester or amide. For example, pharmaceutically acceptable esters (of carboxylic acids of compounds of the invention) that may be mentioned include optionally substituted C₁₋₆ alkyl, C₅₋₁₀ aryl and/or C₅₋₁₀ aryl-C₁₋₆ alkyl-esters. Pharmaceutically acceptable amides (of carboxylic acids of compounds of the invention) that may be mentioned include those of the formula —C(O)N(R²¹)R^{z2}, in which R^{z1} and R^{z2} independently represent optionally substituted C₁₋₆ alkyl, C₅₋₁₀ aryl, or C₅₋₁₀ aryl-C₁₋₆ alkylene. Preferably, C₁₋₆ alkyl groups that may be mentioned in the context of such pharmaceutically acceptable esters and amides are not cyclic, e.g. linear and/or branched.

Further compounds of the invention that may be mentioned include carbamate, carboxamido or ureido derivatives, e.g. such derivatives of existing amino functional groups.

For the purposes of this invention, therefore, prodrugs of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. I-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Scheme 1 and/or in the Examples herein below, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

Unless otherwise stated, the term C_{1-q} alkylene (where q is the upper limit of the range) defined herein may be straight-chain or, when there is a sufficient number of carbon atoms, be saturated or unsaturated (so forming, for example, an alkenylene or alkynylene linker group).

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Heterocycloalkyl mentioned herein may be stated to be specifically monocyclic or bicyclic.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of cycloalkyl or heterocycloalkyl groups) refers to monocyclic or bicyclic groups in which two non-adjacent atoms are linked by either an alkylene or heteroalkylene chain (as appropriate).

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydro-naphthyl. The point of attachment of aryl groups may be via any atom of the ring system. For example, when the aryl group is polycyclic the point of attachment may be via any atom including an atom of a non-aromatic ring. However, when aryl groups are polycyclic (e.g. bicyclic or tricyclic), they are preferably linked to the rest of the molecule via an aromatic ring. When aryl groups are polyyclic in which there is a non-aromatic ring present, then that non-aromatic ring may be substituted by one or more ═O group.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group.

It may be specifically stated that the heteroaryl group is monocyclic or bicyclic. In the case where it is specified that the heteroaryl is bicyclic, then it may be consist of a five-, six- or seven-membered monocyclic ring (e.g. a monocyclic heteroaryl ring) fused with another a five-, six- or seven-membered ring (e.g. a monocyclic aryl or heteroaryl ring).

Heteroatoms that may be mentioned include phosphorus, silicon, boron and, preferably, oxygen, nitrogen and sulfur.

For the avoidance of doubt, where it is stated herein that a group (e.g. a C₁₋₁₂ alkyl group) may be substituted by one or more substituents (e.g. selected from E⁶), then those substituents (e.g. defined by E⁶) are independent of one another. That is, such groups may be substituted with the same substituent (e.g. defined by E⁶) or different substituents (defined by E⁶).

For the avoidance of doubt, in cases in which the identity of two or more substituents in a compound of the invention may be the same, the actual identities of the respective substituents are not in any way interdependent. For example, in the situation in which there is more than one e.g. B¹ to B⁴ or E¹ to E⁸ (such as E⁵) substituent present, then those B¹ to B⁴ or E¹ to E⁸ (e.g. E⁵) substituents may be the same or different. Further, in the case where there are e.g. B¹ to B⁴ or E¹ to E⁸ (such as E⁵) substituents present, in which one represents —C(═Y)OR^{10a} (or e.g. —OR²⁰, as appropriate) and the other represents —C(O)R^{10a} (or e.g. —C(O)₂R²⁰, as appropriate), then those R^{10a} or R²⁰ groups are not to be regarded as being interdependent. Also, when e.g. there are two —OR^{10a} substituents present, then those —OR^{10a} groups may be the same or different (i.e. each R^{10a} group may be the same or different). Further, each of the integers mentioned (e.g. each E⁴) are independent of one another.

For the avoidance of doubt, when a term such as “E¹ to E⁸” is employed herein, this will be understood by the skilled person to mean E¹, E², E³, E⁴, E^{4a}, E⁵, E⁶, E⁷ and E⁸, inclusively. Similarly, the term “B¹ to B⁴” as employed herein will be understood to mean B¹, B^{1a}, B², B^{2a}, B³, B^{3a}, B⁴ and B^{4a}, inclusively.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

In a further embodiment of the invention:

Q¹ may represent: —C(═Y)N(R^{10a})—OR^{11c}, in which R^{10a} is as hereinbefore defined, and R^{11c} represents C₁₋₁₂ alkyl, heterocycloalkyl (which latter two groups are optionally substituted by one or more substituents selected from ═O, ═S, ═N(R²⁰) and E⁶), aryl or heteroaryl (which latter two groups are optionally substituted by one or more substituents selected from E⁷);
Q⁴ and Q⁵ may represent —C(═Y)N(R²⁰)—R^{21a}, in which R²⁰ is as hereinbefore defined, and R^{21a} represents C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y)—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰R⁵¹, —S(O)₂R⁵⁰, —SR⁵⁰, —S(O)R⁵⁰, C₁₋₆ alkyl (optionally substituted by one or more fluoro atoms), heterocyclalkyl, aryl or heteroaryl (which latter three groups are optionally substituted by one or more substituents selected from halo, —OR⁶⁰ and —N(R⁶¹)R⁶²), in which R⁶⁰, R⁶¹ and R⁶² are as hereinbefore defined; alkyl groups or heterocycloalkyl groups mentioned herein may also be (additionally) substituted by ═S or ═N—R^{10a} (although they are preferably not), in which R^{10a} is as hereinbefore defined.

The skilled person will appreciate that the bicyclic core of the compounds of the invention (containing A₁, A₄, A₄ₐ and A₅) is aromatic. It is further stated herein that at least one of A₄ and A₄ₐ does not represent N, i.e. that at least one of C(R^{1a}) or C(R^{1b}) is present. Both C(R^{1a}) and C(R^{1b}) may also be present.

Compounds of the invention that may be mentioned include those in which either:

(i) R^{1b} is present (i.e. A₄ₐ reprsents C(R^{1b})) and represents —C(═Y)N(R^{10a})R^{11a} or —C(═Y)—R^{10a} and R^{1a} (if present) represents hydrogen or Q¹ as hereinbefore defined (this is the preferred option); or
(ii) R^{1a} is present (i.e. A₄ reprsents C(R^{1a})) represents —C(═Y)N(R^{10a})R^{11a} or —C(═Y)—R^{10a} and either R^{1b} is not present (i.e. A₄ₐ represents N) or, preferably, R^{1b} is present (i.e. A₄ represents C(R^{1b})) and represents hydrogen or Q¹, in which Q¹ represents halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, aryl or heteroaryl (which latter two groups are optionally substituted by one or more substituents selected from E^{4a}).

It is preferred that when R^{1a} is present and represents —C(═Y)N(R^{10a})R^{11a} or —C(═Y)—R^{10a}, then R^{1b} is either not present (i.e. A₄ₐ represents N) or, if present, then R^{1b} does not represent alkyl or heterocycloalkyl (both of which may be optionally substituted as defined herein). In this instance, R^{1b} (when present) preferably represents hydrogen or Q¹ in which Q¹ preferably represents halo, —CN, —NO₂, —N(R^{10a})R^{11a}, —OR^{10a}, —C(═Y)—R^{10a}, C(═Y)—OR^{10a}, —C(═Y)N(R^{10a})R^{11a}, —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a} or —S(O)₂N(R^{10a})R^{11a}.

It is also preferred that when R^{1b} is present and represents —C(═Y)N(R^{1a})R^{11a} or —C(═Y)—R^{10a}, then R^{1a} (if present) represents hydrogen or Q¹, in which Q¹ preferably represents halo, —CN, —NO₂, —N(R^{10a})R^{11a}, —OR^{10a}, —C(═Y)R^{10a}, —C(═Y)—OR^{10a}, —C(═Y)N(R^{10a})R^{11a}, —N(R^{12a})C(═Y)R^{11a}, —N(R^{12a})C(═Y)N(R^{10a})R^{11a}, —NR^{12a}S(O)₂R^{10a}, —NR^{12a}S(O)₂N(R^{10a})R^{11a} or —S(O)₂N(R^{10a})R^{11a}.

Preferably, when R^{1a} is present that represents the requisite —C(═Y)N(R^{10a})R^{11a} or —C(═Y)—R^{10a} moiety, then R^{1b} is either not present (i.e. A₄ₐ represents N) or R^{1b} is present (i.e. A₄ₐ represents C(R^{1b})) and preferably represents hydrogen or Q¹ (e.g. halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —N(R^{12a})C(═Y)R^{11a}, —N(R^{12a})C(═Y)N(R^{10a})R^{11a}, —NR^{12a}S(O)₂R^{10a}, —NR^{12a}S(O)₂N(R^{10a})R^{11a} and —S(O)₂N(R^{10a})R^{11a}, in which R^{10a}, R^{11a} and R^{12a} are as hereinbefore defined, provided that they are not linked together). In this instance, R^{1b} preferably represents hydrogen (and Q¹, in these instances, preferably represents halo, —CN or —NO₂).

Preferably, it is R^{1b} that is present and represents the requisite —C(═Y)N(R^{10a})R^{11a} or —C(═Y)—R^{10a} moiety, and either R^{1a} is not present or represents hydrogen or a substituent selected from halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —N(R^{12a})C(═Y)R^{11a}, N(R^{12a})C(═Y)N(R^{10a})R^{11a}, —NR^{12a}S(O)₂R^{10a}, —NR^{12a}S(O)₂N(R^{10a})R^{11a} and —S(O)₂N(R^{10a})R^{11a} (in which R^{10a} and R^{11a} are preferably not linked together).

Other preferred compounds of the invention that may be mentioned include those in which:

Q¹ represents:
halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —OC(═Y)—R^{10a}, —OC(═Y)—OR^{10a}, —OC(═Y)N(R^{10a})R^{11a}, —OS(O)₂OR^{11a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, aryl or heteroaryl (which latter two groups are optionally substituted by one or more substituents selected from E^{4a}); or, more preferably, halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —N(R^{12a})C(═Y)R^{11a} or —NR^{12a}S(O)₂R^{10a} (e.g. halo, —CN, —NO₂, —N(R^{10a})R^{11a} or —OR^{10a});
R^{10a} and R^{11a} are preferably not linked together.

Compounds of the invention include those as hereinbefore defined, but provided that when A₄, A₄₀ and A₅ respectively represent C(R^{1a}), C(R^{1b}) and C(R²), then A₁ does not represent N, i.e. the requisite bicycle (containing A₁, A₄, A₄ₐ and A₅) of formula I may not be the following:

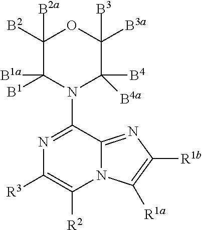

Other compounds of the invention that may be mentioned include those in which: when R^{2a} represents C₁₋₁₂ (e.g. C₁₋₆) alkyl, then that group is preferably substituted by at least one fluoro atom (e.g. it is a perfluoro group);

R^{2a} represents —N(R^{5a})R^{5b}, —N(R^{5c})—C(═Y)—R^{5d}, —N(R^{5e})—C(═Y)—N(R^{5f}), —N(R^{5g})—C(O)—OR^{5h}, —N(R⁵ⁱ)—OR^{5j} or C₁₋₁₂ (e.g. C₁₋₆) alkyl optionally substituted by one or more halo (e.g. fluoro) atoms);
R^{2a} represents —N(R^{5a})R^{5b}, —N(R^{5c})—C(═Y)—R^{5d}, —N(R^{5e})—C(═Y)—N(R^{5f}), —N(R^{5g})—C(O)—OR^{5h} or —N(R⁵ⁱ)—OR^{5j}; and/or
R^{2b} represents R^{2a} (e.g. as defined herein) or, preferably, hydrogen.

Compounds of the invention that may be mentioned include those in which, for example when A₁ and A₄ₐ represent N, A₅ represents C(R²) and A₄ represents C(R^{1a}), then R^{1a} preferably does not represent Q¹ in which Q¹ represents —OR^{10a} (and R^{10a} represents H).

Preferred compounds of the invention include those in which either:
(i) A₁ represents N, A₄ represents C(R^{1a}), A₄ₐ represents C(R^{1b}) and A₅ represents N;
(ii) A₁ represents N, A₄ represents N, A₄ₐ represents C(R^{1b}) and A₅ represents C(R²);
(iii) A₁ represents C(R¹), A₄ represents N, A₄ₐ represents C(R^{1b}) and A₅ represents C(R²),
   i.e. the requisite A₁, A₄, A₄ₐ and A₅-containing bicycle of formula I represents one of the following structures:

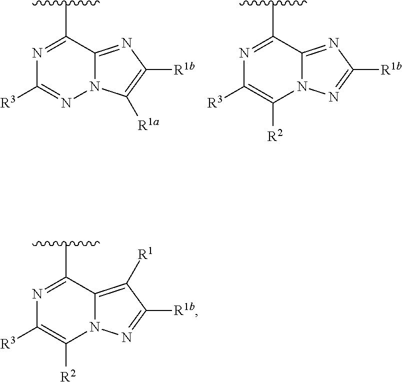

in which R¹, R^{1a}, R^{1b}, R² and R³ are as hereinbefore defined and the squiggly line represents the point of attachment to the requisite (optionally substituted) morpholinyl moiety of the compound of formula I.

The attachment site of the R³ group to the relevant carbon atom of the requisite bicyclic A₁, A₄, A₄ₐ and A₅-containing ring of formula I may preferably be via any carbon of the R³ group (carbon-linked).

It is preferred that R³ represents a fragment of formula IA or IB. Exemplary embodiments of R³ (when R³ represents a fragment of formula IA or IB) include, but are not limited to substituted phenyl, optionally substituted pyridyl (e.g. pyridine-2-one or pyridine-3-ol), pyrimidinyl, triazinyl, pyrrolyl, pyrazolyl, triazolyl, tetrazolyl, thiazolyl, isothiazolyl, oxazolyl, isoxazolyl, pyridazinyl, imidazolyl and pyrazinyl. Exemplary embodiments of R³ (when R³ represents bicyclic aryl) include naphthyl. Exemplary embodiments of R³ (when R³ represents bicyclic heteroaryl) include isatin groups, indolyl (e.g. indolin-2-one), isoindolyl, 1,3-dihydro-indol-2-one-yl, indazolyl, 1-(indolin-1-yl)ethanone-yl, 1H-benzo[d][1,2,3]triazolyl, 1H-pyrazolo[3,4-b]pyridinyl, 1H-pyrazolo[3,4-d]pyrimidinyl, 1H-benzo[d]imidazolyl, 1H-benzo[d]imidazol-2(3H)-one-yl, 1H-pyrazolo[3,4-c]pyridinyl, 1H-pyrazolo[4,3-d]pyrimidinyl, 5H-pyrrolo[3,2-d]pyrimidinyl, 2-amino-1H-purin-6(9H)-one-yl, quinolinyl, quinazolinyl, quinoxalinyl, isoquinolinyl, isoquinolin-1(2H)-one-yl, 3,4-dihydroisoquinolin-1(2H)-one-yl, 3,4-dihydroquinolin-2(1H)-one-yl, quinazolin-2(1H)-one-yl, quinoxalin-2(1H)-one-yl, 1,8-naphthyridinyl, pyrido[3,4-d]pyrimidinyl, pyrido[3,2-b]pyrazinyl, 1,3-dihydro benzimidazolone-yl, benzimidazolyl, benzothiazolyl and benzothiadiazolyl. Most preferred R³ groups include those of fragment IA, for instance phenyl, pyridyl and especially pyrimidinyl (e.g. 5-pyrimidinyl).

When R³ represents a bicyclic heteroaryl group, then it may represent any one of the following structures:

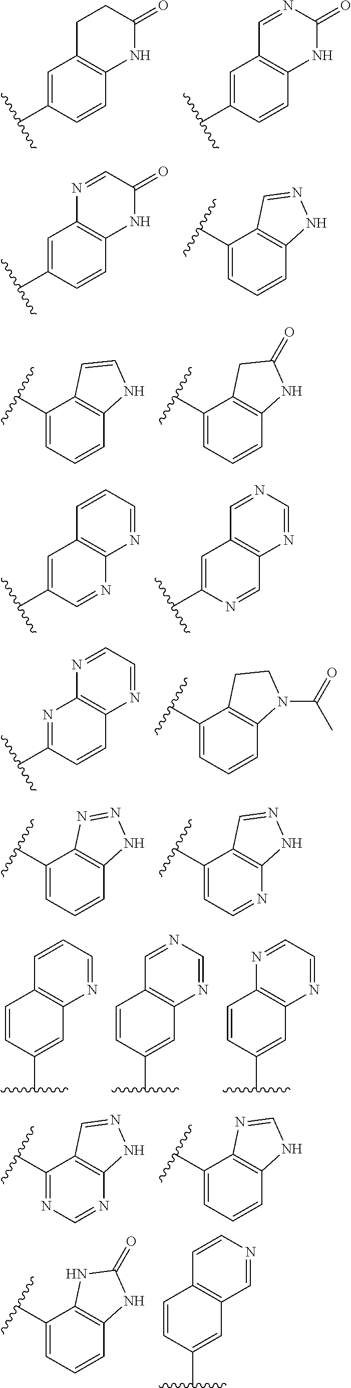

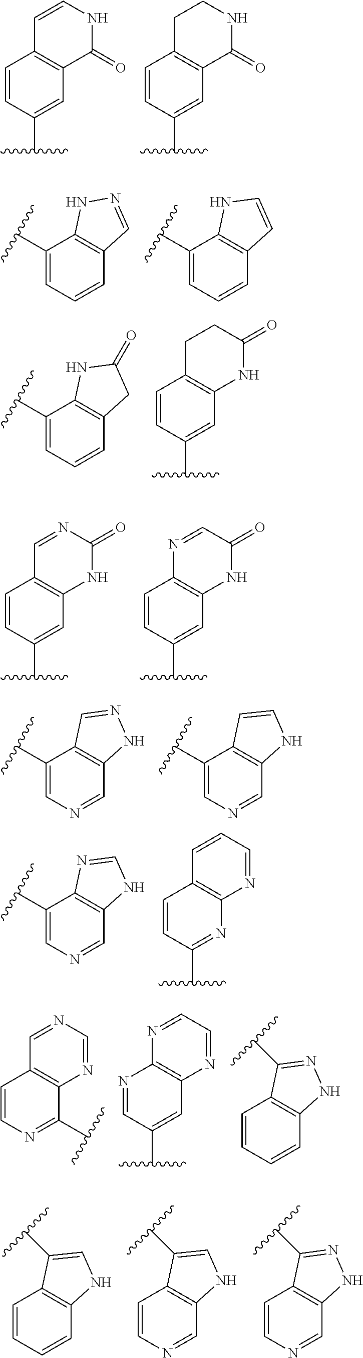

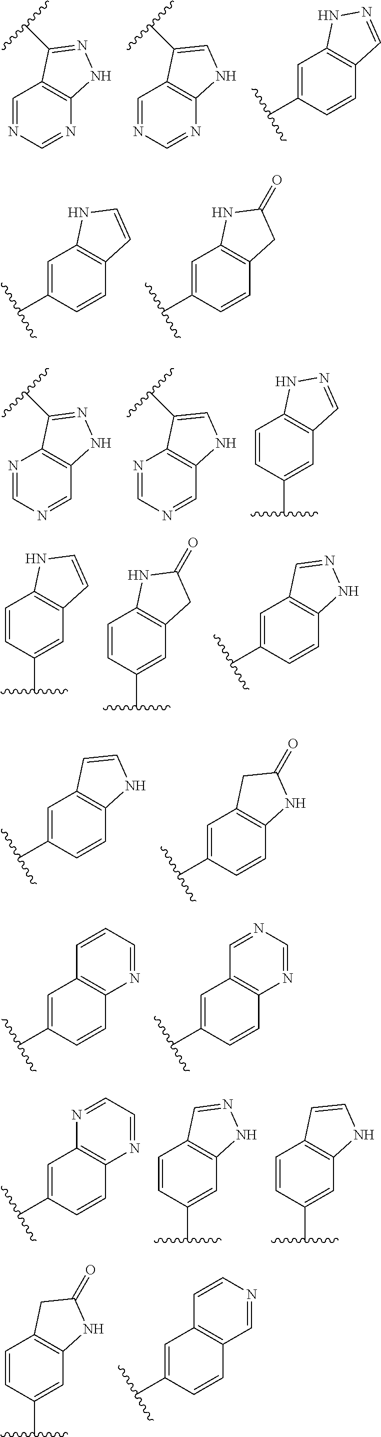

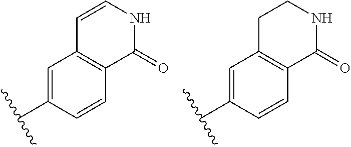 [SMILES: *c1ccc2c(=O)[nH]ccc2c1 ; *c1ccc2c(c1)CCNC2=O]

Compounds of the invention that are preferred include those in which:

when R³ represents aryl (e.g. phenyl), then that group is substituted by one substituent (as defined herein by R^{2a}) and optionally (a) further substituent(s) (e.g. by a further one or two substituents as defined herein by R^{2b});
when R³ represents substituted aryl (e.g. phenyl), then the substituent may be situated at the 2-, 3-, 4-, 5- or 6-position of the phenyl ring (typically one substituent is situated at position 3 or 4, i.e. at the position corresponding to X₂, X₃ or X₄; most preferably the substituent is situated at the 4-position, i.e. X³ represents C(R^{2a}) in which R^{2a} is a substituent as defined herein);
R^{2a} may represent (e.g. when attached to a phenyl ring) a —OH moiety, for instance the —OH group is typically situated at the 3- or 4-position of the phenyl ring, so forming a 3-hydroxyphenyl or 4-hydroxyphenyl group or an isostere thereof, which is unsubstituted or substituted; an isostere as used herein is a functional group which possesses binding properties which are the same as, or similar to, the 3-hydroxyphenyl or 4-hydroxyphenyl group in the context of the compounds of the invention; isosteres of 3-hydroxyphenyl and 4-hydroxyphenyl groups are encompassed within the definition of R³ (and R^{2a} may also represent another moiety as defined herein);
when R³ represents heteroaryl, it is unsubstituted or substituted (when substituted, it may be substituted by one or more (e.g. one or two) substitutents selected from those listed in respect of R^{2a} and R^{2b});
when R³ is a phenyl group typically the substituents are selected from —OR^{5k} (e.g. —OH) and, especially —N(R^{5a})R^{5b} (e.g. NH₂).

Preferred compounds of the invention include those in which:

R³ preferably represents a fragment of formula IA;
any one of X₁, X₂, X₃, X₄ and X₅ (e.g. X₃) represents C(R^{2a}), and the remaining groups (e.g. X₁, X₂, X₄ and X₅) independently represent C(R^{2b}), or X² and X⁴ may alternatively and independently represent N;
R^{2a} represents C₁₋₄ (e.g. C₁₋₂) alkyl substituted by one or more fluoro atoms or R^{2a} represents —N(R^{5c})—C(═Y)—R^{5d} or, preferably, —N(R^{5a})R^{5b};
when R^{2a} represents alkyl substituted by one or more fluoro atoms, then it is preferably a perfluoro group (e.g. trifluoromethyl);

each R^{2b} independently represents halo or, preferably, hydrogen; each R^{5a}, R^{5b}, R^{5c}, R^{5d}, R^{5e}, R^{5f}, R^{5g}, R^{5h}, R⁵ⁱ, R^{5j}, R^{5k}, R^{5m} and R⁵ⁿ (e.g. R^{5a}, R^{5b}, R^{5c} and R^{5d}) independently represent C₁₋₄ (e.g. C₁₋₂) alkyl or, preferably, hydrogen;

when R³ represents a fragment of formula IB, then any one or two of X₆, X₇, X₈ and X₉ represents a heteroatom selected from nitrogen, oxygen and sulfur and the other two or three independently represent C(R^{2b});
when R³ represents bicyclic heteroaryl (e.g. a 8-, 9- or 10-membered heteroaryl group), then that group preferably consists of a 5- or 6-membered ring fused to another 5- or 6-membered ring (in which either one of those rings may contain one or more (e.g. four, or, preferably one to three) heteroatoms), in which the total number of heteroatoms is preferably one to four, and which ring is optionally substituted by one or more (e.g. two or, preferably, one) substituent(s) selected from E⁵ (and, if there is a non-aromatic ring present in the bicyclic heteroaryl group, then such a group may also be substituted by one or more (e.g. one) ═O groups).

Further preferred compounds of the invention include those in which:

each R^{10a}, R^{11a}, R^{10b}, R^{11b} and R^{12a} independently represent, on each occasion when used herein, hydrogen or C₁₋₁₂ (e.g. C₁₋₆) alkyl (which latter group is optionally substituted by one or more substituents selected from ═O and E⁶); or any relevant pair of R^{10a} and R^{11a} and/or R^{10b} and R^{11b} may, when attached to the same nitrogen atom, be linked together to form (along with the requisite nitrogen atom to which they are attached) a 3- to 12- (e.g. 4- to 12-) membered ring, optionally containing one or more (e.g. one to three) double bonds, and which ring is optionally substituted by one or more substituents selected from E⁸ and ═O;
each of E¹, E², E³, E⁴, E^{4a}, E⁵, E⁶, E⁷ and E⁸ independently represents, on each occasion when used herein, Q⁴ or C₁₋₆ alkyl (e.g. C₁₋₃) alkyl optionally substituted by one or more substituents selected from ═O and Q⁵;
each Q⁴ and Q⁵ independently represent halo, —CN, —NO₂, —N(R²⁰)R²¹, —OR²⁰, —C(═Y)—R²⁰, —C(═Y)—OR²⁰, —C(═Y)N(R²⁰)R²¹, —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —S(O)₂R²⁰, —SR²⁰, —S(O)R²⁰ or C₁₋₆ alkyl optionally substituted by one or more fluoro atoms (and each Q⁵ more preferably represents halo, such as fluoro);
any two E¹, E², E³, E⁴, E⁵, E⁶, E⁷ or E⁸ groups may be linked together, but are preferably not linked together;
each R²⁰, R²¹, R²² and R²³ independently represent, on each occasion when used herein, aryl (e.g. phenyl; preferably unsubstituted, but which may be substituted by one to three J⁵ groups) or, more preferably, hydrogen or C₁₋₆ (e.g. C₁₋₃) alkyl optionally substituted by one or more substituents selected from ═O and J⁴; or
any pair of R²⁰ and R²¹, may, when attached to the same nitrogen atom, be linked together to form a 4- to 8-membered (e.g. 5- or 6-membered) ring, optionally containing one further heteroatom selected from nitrogen and oxygen, optionally containing one double bond, and which ring is optionally substituted by one or more substituents selected from J⁶ and ═O;
each J¹, J², J³, J⁴, J⁵ and J⁶ independently represent C₁₋₆ alkyl (e.g. acyclic C₁₋₃ alkyl or, e.g. in the case of J⁴, C₃₋₅ cycloalkyl) optionally substituted by one or more substituents selected from ═O and Q⁸, or, more preferably, such groups independently represent a substituent selected from Q⁷;
each Q⁷ and Q⁸ independently represents a substituent selected from fluoro, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —NR⁵²S(O)₂R⁵⁰, —S(O)₂R⁵⁰ or C₁₋₆ alkyl optionally substituted by one or more fluoro atoms;
each R⁵⁰, R⁵¹, R⁵² and R⁵³ substituent independently represents, on each occasion when used herein, hydrogen or C₁₋₆ (e.g. C₁₋₃) alkyl optionally substituted by one or more substituents selected from fluoro;
when any relevant pair of R⁵⁰, R⁵¹ and R⁵² are linked together, then those pairs that are attached to the same nitrogen atom may be linked together (i.e. any pair of R⁵⁰ and R⁵¹), and the ring so formed is preferably a 5- or 6-membered ring, optionally containing one further nitrogen or oxygen heteroatom, and which ring is optionally substituted by one or more substituents selected from ═O and C₁₋₃ alkyl (e.g. methyl);
R⁶⁰, R⁶¹ and R⁶² independently represent hydrogen or C₁₋₃ (e.g. C₁₋₂) alkyl optionally substituted by one or more fluoro atoms.

Preferred optional substituents (in addition to any requisite substituent defined herein by R^{2a}) on R³ (and, possibly when they represent a substituent other than hydrogen on R¹, R^{1a}, R^{1b} and R² groups) include:

═O (e.g. in the case of alkyl, cycloalkyl or heterocycloalkyl groups);

### —CN;

halo (e.g. fluoro, chloro or bromo);
C₁₋₄ alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl) or substituted with an aryl, heteroaryl or heterocycloalkyl group (which themselves may be substituted with one or more —OR^{z1}, —C(O)R^{z2}, —C(O)OR^{z3}, —N(R^{z4})R^{z5}, —S(O)₂R^{z6}, —S(O)₂N(R^{z7})R^{z8}; —N(R^{z9})—C(O)—R^{z10}, —C(O)—N(R^{z11})R^{z12} and/or —N(R^{z9})—C(O)—N(R^{z10}) substituents; aryl (e.g. phenyl), if appropriate (e.g. when the substitutent is on an alkyl group, thereby forming e.g. a benzyl group);

### —OR^{z1};

### —C(O)R^{z2};

### —C(O)OR^{z3};

### —N(R^{z4})R^{z5};

### —S(O)₂R^{z6};

### —S(O)₂N(R^{z7})R^{z8};

### —N(R^{z9})—C(O)—R^{z10};

### —C(O)—N(R^{z11})R^{z12};

### —N(R^{z9})—C(O)—N(R^{z10});

wherein each R^{z1} to R^{z12} independently represents, on each occasion when used herein, H or C₁₋₄ alkyl (e.g. ethyl, n-propyl, t-butyl or, preferably, n-butyl, methyl, isopropyl or cyclopropylmethyl (i.e. a part cyclic alkyl group)) optionally substituted by one or more halo (e.g. fluoro) groups (so forming e.g. a trifluoromethyl group). Further, any two R^{z} groups (e.g. R^{z4} and R^{z5}), when attached to the same nitrogen heteroatom may also be linked together to form a ring such as one hereinbefore defined in respect of corresponding linkage of R^{10a} and R^{11b} or R^{10b} and R^{11b} groups.

Preferred compounds of the invention include those in which:

R² represents hydrogen or a substituent selected from —N(R^{10b})R^{11b} and, preferably, halo (e.g. chloro, bromo or iodo) and —CN (but R² most preferably represents hydrogen);
B¹, B^{1a}, B², B^{2a}, B³, B^{3a}, B⁴ and B^{4a} independently represent hydrogen, C₁₋₆ (e.g. C₁₋₃) alkyl optionally substituted by one or more substituents selected from ═O and E¹, any two of these together form a ═O substituent on the morpholinyl ring, or, any two B¹, B^{1a}, B², B^{2a}, B³, B^{3a}, B⁴ and B^{4a} substituents when linked together, may form a linkage, for example between a B² or B^{2a} substituent and a B³ or B^{3a} substituent to form a further ring, e.g. a five membered ring such as the one depicted below:

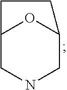

each E¹, E², E³, E⁴, E^{4a}, E⁵, E⁶, E⁷ and E⁸ independently represents C₁₋₁₂ alkyl optionally substituted by one or more substituents selected from ═O and Q⁵, or, preferably (each E¹ to E⁸ independently represents) Q⁴;
each R²⁰, R²¹, R²² and R²³ (e.g. each R²⁰ and R²¹) independently represents heteroaryl, preferably, aryl (e.g. phenyl) (which latter two groups are optionally substituted by one or more substituents selected from J⁵), or, more preferably, hydrogen or C₁₋₆ (e.g. C₁₋₄) alkyl optionally substituted by one or more substituents selected from ═O and J⁴; or
any relevant pair of R²⁰, R²¹ and R²² (e.g. R²⁰ and R²¹) may (e.g. when both are attached to the same nitrogen atom) may be linked together to form a 3- to 8- (e.g. 4- to 8-) membered ring, optionally containing a further heteroatom, and optionally substituted by one or more substituents selected from ═O and J⁶;
each J¹, J², J³, J⁴, J⁵ and J⁶ independently represent C₁₋₆ alkyl (e.g. C₁₋₃ acyclic alkyl or C₃₋₅ cycloalkyl) optionally substituted by one or more substituents selected from Q⁸, or, J¹ to J⁶ more preferably represent a substituent selected from Q⁷;
each Q⁷ and Q⁸ independently represent halo, —N(R⁵⁰)R⁵¹, —OR⁵⁰, —C(═Y^{a})—OR⁵⁰, —C(═Y^{a})—R⁵⁰, —S(O)₂R⁵⁰ or C₁₋₃ alkyl optionally substituted by one or more fluoro atoms;
each R⁵⁰, R⁵¹, R⁵² and R⁵³ independently represents hydrogen or C₁₋₆ (e.g. C₁₋₄) alkyl optionally substituted by one or more fluoro atoms;
each R⁶⁰, R⁶¹ and R⁶² independently represents hydrogen or C₁₋₂ alkyl (e.g. methyl).

More preferred compounds of the invention include those in which:

R² represents hydrogen, chloro, bromo, iodo or —CN;
each R^{10a}, R^{11a}, R^{10b}, R^{11b} and R^{12a} independently represents hydrogen or C₁₋₄ (e.g. C₁₋₃) alkyl, which alkyl group may by substituted by one or more substituents selected from ═O and E⁶ (but which alkyl group is more preferably unsubstituted); or
any relevant pair of R^{10a} and R^{11a} and/or R^{10b} and R^{11b}, may be linked together to form a 5- or, preferably, a 6-membered ring, optionally containing a further heteroatom (preferably selected from nitrogen and oxygen), which ring is preferably saturated (so forming, for example, a piperazinyl or morpholinyl group), and optionally substituted by one or more substituents selected from ═O and E⁸ (which E⁸ substituent may be situated on a nitrogen heteroatom; and/or E⁸ is preferably halo (e.g. fluoro) or C₁₋₃ alkyl optionally substituted by one or more fluoro atoms);
each E¹, E², E³, E⁴, E^{4a}, E⁵, E⁶, E⁷ and E⁸ independently represent C₁₋₄ alkyl optionally substituted by one or more Q⁵ substituents, or, each of these preferably represent a substituent selected from Q⁴;
Q⁴ and Q⁵ independently represent halo (e.g. fluoro), —OR²⁰, N(R²⁰)R²¹, —C(═Y)OR²⁰, —C(═Y)N(R²⁰)R²¹, —NR²²S(O)₂R²⁰, heterocycloalkyl, aryl, heteroaryl (which latter three groups are optionally substituted with one or more substitutents selected from J² or J³, as appropriate) and/or C₁₋₆ alkyl (e.g. C₁₋₃ alkyl) optionally substituted by one or more fluoro atoms;
each Y represents, on each occasion when used herein, ═S, or preferably ═O;
each R²⁰, R²¹, R²² and R²³ (e.g. each R²⁰ and R²¹) independently represents hydrogen or C₁₋₄ (e.g. C₁₋₃) alkyl (e.g. tert-butyl, ethyl, methyl or a part cyclic group such as cyclopropylmethyl) optionally substituted (but preferably unsubstituted) by one or more (e.g. one) J⁴ substituent(s); or
any relevant pair of R²⁰, R²¹ and R²² (e.g. R²⁰ and R²¹) may (e.g. when both are attached to the same nitrogen atom) be linked together to form a 5- or, preferably, a 6-membered ring, optionally containing a further heteroatom (preferably selected from nitrogen and oxygen), which ring is preferably saturated (so forming, for example, a piperazinyl or morpholinyl group), and optionally substituted by one or more substituents selected from ═O and J⁶ (which J⁶ substituent may be situated on a nitrogen heteroatom);
R²² represents C₁₋₃ alkyl or, preferably, hydrogen;
each J¹, J², J³, J⁴, J⁵ and J⁶ independently represent a substituent selected from Q⁷, or J¹ to J⁶ (e.g. J⁴) represents C₁₋₆ alkyl (e.g. C₃₋₅ cycloalkyl);
each Q⁷ and Q⁸ independently represent —C(═Y^{a})—OR⁵⁰, —C(═Y^{a})—R⁵⁰, —S(O)₂R⁵⁰ or C₁₋₃ alkyl optionally substituted by one or more fluoro atoms;
each Y^{a} independently represents ═S or, preferably, ═O;
each R⁵⁰ independently represents C₁₋₄ alkyl (e.g. tert-butyl or methyl).

Preferred R³ groups of the compounds of the compounds of the invention include substituted phenyl, optionally substituted indazolyl (e.g. 4-indazolyl), pyrimidinyl (e.g. 5-pyrimidinyl), azaindolyl (e.g. azaindol-5-yl), indolyl (e.g. 5-indolyl or 4-indolyl) and pyridyl (e.g. 3-pyridyl). Most preferred R³ groups include optionally substituted (but preferably substituted by at least one substituent, e.g. as defined by R^{2a}) pyrimidinyl (e.g. in which X² and X⁴ represent N, X³ represents C(R^{2a}) and X¹ and X⁵ independently represent C(R^{2b})).

Preferred compounds of the invention include those in which:

R² represents hydrogen or halo (e.g. chloro);
R³ represents substituted phenyl (as defined by fragment IA), optionally substituted 5- or 6-membered monocyclic heteroaryl (as defined by fragment IA or fragment IB) or a 9- or 10-membered bicyclic heteroaryl group (which heteroaryl groups may contain one to four, e.g. 3 or, preferably, 1 or 2, heteroatoms preferably selected from nitrogen, oxygen and sulfur) both of which heteroaryl moieties are optionally substituted by one or more (e.g. two, or, preferably, one) substituent(s) selected from R^{2b} (when it represents a substituent, i.e. it is other than hydrogen), R^{2a} and/or E⁵ (e.g. —CF₃, preferably, —OH, —OCH₃ and/or —N(R^{5a})R^{5b} or —N(R²⁰)R²¹ as appropriate (e.g. —NH₂));
E¹ to E⁸ independently represent C₁₋₆ (e.g. C₁₋₃, such as methyl) alkyl optionally substituted by one or more Q⁵ substituents, or, preferably, Q⁴;
Q⁴ represents —OR²⁰, —N(R²⁰)R²¹, —S(O)₂R²⁰, heterocycloalkyl (e.g. a 4- to 6-membered ring, containing preferably one or two heteroatoms selected from nitrogen and oxygen; which heterocycloalkyl group may be substituted but is preferably unsubstituted), aryl (e.g. phenyl; optionally substituted with two or, preferably, one substituent selected from J³) or heteroaryl (e.g. a 5- or 6-membered monocyclic heteroaryl group preferably containing one or two heteroatoms preferably selected from nitrogen, oxygen and sulfur; which group may be substituted, but is preferably unsubstituted);
Q⁵ represents halo (e.g. fluoro);
Y represents ═O;
R²⁰ and R²¹ independently represent hydrogen, C₁₋₃ alkyl (e.g. methyl or ethyl), which latter group is optionally substituted by one or more (e.g. one) substituent(s) selected from J⁴;
when there is a —N(R²⁰)R²¹ moiety present, then one of R²⁰ and R²¹ represents hydrogen, and the other represents hydrogen or C₁₋₃ alkyl (e.g. methyl or ethyl), which latter group is optionally substituted by one or more (e.g. one) substituent(s) selected from J⁴;
J³ represents Q⁷;
J⁴ represents C₁₋₆ alkyl, such as C₃₋₆ alkyl (especially C₃₋₆ cycloalkyl, such as cyclopropyl);
Q⁷ represents —S(O)₂R⁵⁰;
R⁵⁰ represents C₁₋₃ alkyl (e.g. methyl).

Particularly preferred compounds of the invention include those in which:

R² represents hydrogen or chloro;
R³ represents pyrimidinyl (e.g. 5-pyrimidinyl, such as 2-amino-5-pyrimidinyl (such as 2-NH₂-pyrimidin-5-yl);
B¹, B^{1a}, B², B^{2a}, B³, B^{3a}, B⁴ and B^{4a} independently represent hydrogen.

Particularly preferred compounds of the invention include those in which:

A₄ₐ represents C(R^{1b});
the requisite 6,5-fused bicycle represents:
(i) A₁ represents N, A₄ represents C(R^{1a}), A₄ₐ represents C(R^{1b}) and A₅ represents N;
(ii) A₁ represents N, A₄ represents N, A₄ₐ represents C(R^{1b}) and A₅ represents C(R²);
(iii) A₁ represents C(R¹), A₄ represents N, A₄ₐ represents C(R^{1b}) and A₅ represents C(R²),
   in which the bicycles (i) and (iii) are particularly preferred;
   one of R^{1a} and R^{1b} (preferably R^{1b}) is present, which represents —C(═Y)N(R^{10a})R^{11a} or —C(═Y)—OR^{10a}, and when the other (e.g. R^{1a}) is present, then it preferably represents hydrogen or Q¹ (especially hydrogen);
   Q¹ represents halo, —CN, —NO₂, —N(R^{10a})R^{11a} or —OR^{10a} (in which R^{10a} and R^{11a} preferably, and independently, represent hydrogen or C₁₋₄ (e.g. C₁₋₂) alkyl);
   Y represents ═O;
   when R^{1a} or R^{1b} represents —C(═Y)N(R^{10a})R^{11a}, then preferably R^{10a} and R^{11b} independently represent hydrogen, C₁₋₄ (e.g. C₁₋₂) alkyl (optionally (and preferably) substituted by one or more (e.g. one) substituent(s) selected from E⁵) or heterocycloalkyl (e.g. a 5- or, particularly, 6-membered heterocycloalkyl group containing one or two heteroatoms (e.g. one oxygen heteroatom, so forming e.g. a tetrahydropyranyl group, e.g. 4-tetrahydropyranyl); and which heterocycloalkyl group is preferably unsubstituted);
   R^{1a} (when/if present) represents hydrogen;
   R³ represents a fragment of formula IA (e.g. pyrimidinyl, such as 5-pyrimidinyl);
   X² and X⁴ independently represent N;
   X¹ and X⁵ independently represent C(R^{2b});
   X³ represents C(R^{2a});
   each R^{2b} independently represents halo or, preferably, hydrogen;
   R^{2a} represents —N(R^{5a})R^{5b};
   R^{5a} and R^{5b} independently represent hydrogen;
   R^{10a} and R^{11a} independently represent hydrogen or C₁₋₄ (e.g. C₁₋₂) alkyl or heterocycloalkyl (e.g. a 5- or, preferably, 6-membered heterocycloalkyl group), which latter two groups are optionally (and preferably) substituted by one or more (e.g. one) substituent(s) selected from E⁵;
   at least one of R^{10a} and R^{11b} (when attached to the same nitrogen atom) represents hydrogen;
   E⁵ represents Q⁴;
   Q⁴ represents —OR²⁰:
   R²⁰ represents C₁₋₄ (e.g. C₁₋₂) alkyl (e.g. methyl).

Other particularly preferred compounds of the invention include those in which:

when R^{1a} or R^{1b} represents —C(═Y)N(R^{10a})R^{11a}, then preferably R^{10a} and R^{11a} independently represent hydrogen, C₁₋₆ (e.g. C₁₋₃) alkyl (optionally substituted by one or more (e.g. one) substituent(s) selected from E⁵; and which alkyl group may be cyclic, e.g. a C₅₋₆ cycloalkyl group, also optionally substituted) or heterocycloalkyl (e.g. a 5- or, particularly, 6-membered heterocycloalkyl group containing one or two heteroatoms (e.g. one oxygen or nitrogen heteroatom, so forming e.g. a tetrahydropyranyl or piperidinyl group, e.g. 4-tetrahydropyranyl or 4-piperidinyl), or, R^{10a} and R^{11a} may be linked together to form a 5- or 6-membered ring, preferably saturated and unsubstituted, e.g. a pyrrolidinyl; R^{10a} and R^{11a} independently represent hydrogen or C₁₋₄ (e.g. C₁₋₂) alkyl or heterocycloalkyl (e.g. a 5- or, preferably, 6-membered heterocycloalkyl group), which latter two groups are optionally substituted by one or more (e.g. one) substituent(s) selected from E⁵, or, R^{10a} and R^{11a} are optionally linked as hereinbefore defined;
at least one of R^{10a} and R^{11a} (when attached to the same nitrogen atom, and when R^{10a} and R^{11a} are not linked together) represents hydrogen;
E⁵ represents Q⁴;
Q⁴ represents —OR²⁰ or —N(R²⁰)R²¹;
R²⁰ represents hydrogen or C₁₋₄ (e.g. C₁₋₂) alkyl (e.g. methyl).

Particularly preferred R³ groups include 2-amino-5-pyrimidinyl (e.g. 2-N—[—CH₂-(4-OCH₃)phenyl]₂-5-pyrimidinyl and, preferably, 2-NH₂-5-pyrimidinyl). Particularly preferred R^{1a} or R^{1b} (one of which is necessarily present, e.g. R^{1b}) groups include —C(O)—NH₂, —C(O)N(H)—CH₂CH₂—OCH₃ and —C(O)—N(H)-[4-tetrahydropyranyl].

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) reaction of a compound of formula II,

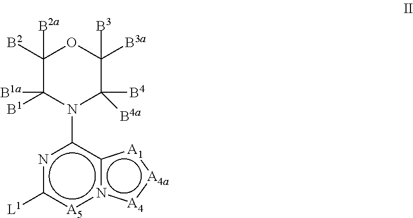

wherein L¹ represents a suitable leaving group, such as iodo, bromo, chloro, a sulfonate group (e.g. —OS(O)₂CF₃, —OS(O)₂CH₃ or —OS(O)₂PhMe), or a sulfide group (e.g. —S—C₁₋₆ alkyl, such as —SCH₃) and A₁, A₄, A₄ₐ, A₅, B¹, B^{1a}, B², B^{2a}, B³, B^{3a}, B⁴ and B^{4a} are as hereinbefore defined, with a compound of formula III,

R³-L² III

wherein L² represents a suitable group such as —B(OH)₂, —B(OR^{wx})₂ or —Sn(R^{wx})₃, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), thereby forming e.g. a pinacolato boronate ester group, (or L² may represent iodo, bromo or chloro, provided that L¹ and L² are mutually compatible) and R³ is as hereinbefore defined. The reaction may be performed, for example in the presence of a suitable catalyst system, e.g. a metal (or a salt or complex thereof) such as Pd, CuI, Pd/C, PdCl₂, Pd(OAc)₂, Pd(Ph₃P)₂Cl₂, Pd(Ph₃P)₄ (i.e. palladium tetrakistriphenylphosphine), Pd₂(dba)₃ and/or NiCl₂ (preferred catalysts include palladium) and a ligand such as PdCl₂(dppf).DCM, t-Bu₃P, (C₆H₁₁)₃P, Ph₃P, AsPh₃, P(o-Tol)₃, 1,2-bis(diphenylphosphino)ethane, 2,2′-bis(di-tert-butyl-phosphino)-1,1′-biphenyl, 2,2′-bis(diphenylphosphino)-1,1′-bi-naphthyl, 1,1′-bis(diphenyl-phosphino-ferrocene), 1,3-bis(diphenylphosphino)propane, xantphos, or a mixture thereof (preferred ligands include PdCl₂(dppf).DCM), together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof; preferred bases include Na₂CO₃ and K₂CO₃) in a suitable solvent such as dioxane, toluene, ethanol, dimethylformamide, dimethoxyethane, ethylene glycol dimethyl ether, water, dimethylsulfoxide, acetonitrile, dimethylacetamide, N-methylpyrrolidinone, tetrahydrofuran or mixtures thereof (preferred solvents include dimethylformamide and dimethoxyethane). When L¹ represents a sulfide (e.g. —SCH₃), then an additive such as CuMeSal (copper(I) 3-methylsalicylate) or CuTC (copper(1)thiophene-2-carboxylate) may also be employed. The reaction may be carried out for example at room temperature or above (e.g. at a high temperature such as at about the reflux temperature of the solvent system). Alternative reaction conditions include microwave irradiation conditions, for example at elevated temperature, e.g. of about 130° C.;
(ii) reaction of a compound of formula IV,

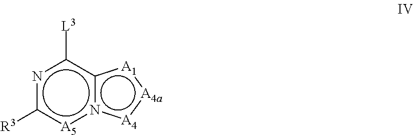

wherein L³ represents a suitable leaving group, such as one hereinbefore defined in respect of L¹ or a sulfone (e.g. —S(O)₂C₁₋₆ alkyl moiety, such as —S(O)₂CH₃) or sulfide (e.g. —S—C₁₋₆ alkyl moiety, such as —SCH₃) and A₁, A₄, A₄ₐ, A₅ and R³ as hereinbefore defined, with a compound of formula V,

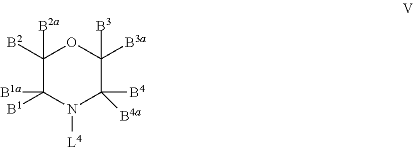

wherein L⁴ may represent hydrogen (so forming an amine group), and B¹, B^{1a}, B², B^{2a}, B³, B^{3a}, B⁴ and B^{4a} are as hereinbefore defined, and the reaction may optionally be performed in the presence of an appropriate metal catalyst (or a salt or complex thereof) such as Cu, Cu(OAc)₂, CuI (or CuI/diamine complex), copper tris(triphenylphosphine)bromide, Pd(OAc)₂, tris(dibenzylideneacetone)-dipalladium(0) (Pd₂(dba)₃) or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). This reaction may be performed at elevated temperature or under microwave irradiation reaction conditions, for example as described in process step (i). The compound of formula IV (e.g. in which L³ is chloro) may be prepared in situ, for example from a compound corresponding to a compound of formula IV, but in which L³ represents —OC₁₋₃ alkyl (e.g. methoxy) by reaction in the presence of e.g. a chlorinating agent (such as POCl₃);
(iii) for compounds of formula I in which (A⁵ represents C(R²) and) R² represents halo (e.g. bromo, iodo or chloro), reaction of a corresponding compound of formula I, in which R² represents hydrogen, with a reagent that is a source of halide ions (a halogenating reagent). For instance, an electrophile that provides a source of iodide ions includes iodine, diiodoethane, diiodotetrachloroethane or, preferably, N-iodosuccinimide, a source of bromide ions includes N-bromosuccinimide and bromine, and a source of chloride ions includes N-chlorosuccinimide, chlorine and iodine monochloride, for instance in the presence of a suitable solvent, such as CHCl₃ or an alcohol (e.g. methanol), optionally in the presence of a suitable base, such as a weak inorganic base, e.g. sodium bicarbonate. Typically, the reaction maybe performed by heating at a convenient temperature, either by conventional heating under reflux or under microwave irradiation;
(iv) for compounds of formula I in which R² (if present; i.e. if A⁵ represents C(R²)) represents a substituent other that hydrogen, or halo (e.g. bromo, iodo or chloro), reaction of a corresponding compound of formula I, in which R² represents halo (e.g. bromo, chloro or iodo), with a compound of formula VI,

R^{2aa}-L⁷ VI

wherein R^{2aa} represents R² as hereinbefore described provided that it does not represent hydrogen or halo, and L⁷ represents a suitable leaving group such as one hereinbefore described in respect of L¹ or L² (see e.g. process step (i); reaction conditions such as those mentioned above may also be employed). Alternatively, the skilled person will appreciate that different reagents and reaction steps may be employed, depending on the particular R^{2aa} substituent required;
(v) for compounds of formula I in which R^{1a} and/or R^{1b} represents —C(O)N(R^{10a})R^{11a}, reaction of a compound of formula VIA,

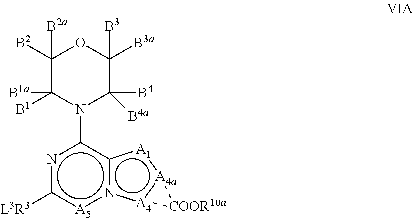

wherein L¹R³ represents either L¹ as hereinbefore defined or R³ as hereinbefore defined, and wherein either A₄ or A₄ₐ represent C(R^{1a}) or C(R^{1b}) respectively, in which either R^{1a} or R^{1b} represents the relevant —COOR^{10a} moiety (i.e. —COOR^{10a} is attached to a carbon atom at either the A₄ or A₄ₐ position), and the other one of A₄ or A₄ₐ represents N or C(R^{1a}) or C(R^{1b}) (as appropriate) in which the other R^{1a} or R^{1b} group represents hydrogen or Q¹ as hereinbefore defined, and B¹, B^{1a}, B², B^{2a}, B³, B^{3a}, B⁴, B^{4a}, A₁, A₄, A₄, R³ and R^{10a} are as hereinbefore defined (R^{10a} is preferably hydrogen or optionally substituted alkyl), with a compound of formula VIB,

HN(R^{10a})R^{11a} VIB

wherein R^{10a} and R^{11a} are as hereinbefore defined, under standard amide coupling reaction conditions, i.e. conditions that promote the formation of an amide from a carboxylic acid (or ester thereof), for example in the presence of a suitable coupling reagent (e.g. 1,1′-carbonyldiimidazole, N,N′-dicyclohexylcarbodiimide, HBTU or the like) or, in the case when R² represents an ester (e.g. —C(O)OCH₃ or —C(O)OCH₂CH₃), in the presence of e.g. trimethylaluminium, or, alternatively a —C(O)OH group may first be activated to the corresponding acyl halide (e.g —C(O)CI, by treatment with oxalyl chloride, thionyl chloride, phosphorous pentachloride, phosphorous oxychloride, or the like), and, in all cases, the relevant compound is reacted with a compound of formula VIB as defined above, under standard conditions known to those skilled in the art (e.g. optionally in the presence of a suitable solvent, suitable base and/or in an inert atmosphere). Alternatively, for coupling with ammonia (e.g. ammonia in alcoholic solution), the coupling reaction may take place in the presence of a suitable solvent (e.g. dichloromethane) in a sealed tube. When reaction takes place on a compound of formula VIA in which R^{10a} represents a substituent other than hydrogen (e.g. optionally substituted alkyl), then the reaction may also take place in a sealed tube. In this case, a solution of Me₃Al (e.g. 2 M in hexanes), or the like, may be added to the amine (of formula VIB), which mixture is in turn added to the compound of formula VIA (thereafter, it may be heated at reflux, then cooled before work up);
(vi) for compounds of formula I in which R^{1a} and/or R^{1b} is present that represents halo or —C(O)OR^{10a} may be prepared from corresponding compounds of formula I in which R^{1a} or R^{1b} (as appropriate) represents hydrogen, which may be reacted in the presence of a suitable base, such as an organometallic base (e.g. an organolithium base, such as t-, s- or n-butyllithium or, preferably a lithium amide base such as diisopropylamide; which deprotonates and/or lithiates at the relevant position), followed by reaction in the presence of an electrophile that is a source of halide ions (e.g. as described in respect of process step (iii)), or CO₂ (to form compounds of formula I in which R^{1a} and/or R^{1b} represent —COOH) or a compound of formula VII,

L⁸-R^{1b1} VII

wherein L⁸ represents a suitable leaving group, such as one hereinbefore defined in respect of L¹ (or another suitable leaving group), and R^{1b1} represents —C(O)OR^{10a} (and R^{10a} is preferably not hydrogen), under standard reaction conditions, for example the deprotonation/lithiation may be performed in an inert atmosphere (e.g. under N₂) in the presence of an anhydrous polar aprotic solvent (such as THF, dimethoxyethane, ethyl ether and the like), which may be performed at below room temperature (e.g. at below 0° C., at temperatures down to −78° C., depending on the strength of the base to be employed), and the subsequent ‘quench’, i.e. reaction with the electrophile (e.g. halide source or compound of formula VII) may also be performed at low temperatures (e.g. at the temperature of the deprotonation/lithiation), which temperature may be raised up to 0° C. (or to rt) to ensure the complete reaction, before the mixture is worked up;
(vii) for compounds of formula I which contain a —C(OH)(H)—C₁₋₁₁ alkyl group (which alkyl group may be substituted by one or more substituents selected from those defined herein, e.g. E³ and ═O; halo; or ═O, ═S and ═N(R²⁰) as appropriate, but is preferably unsubstituted), for example when there is a R¹ and/or R² group present which represent such a —C(OH)(H)—C₁₋₁₁ alkyl group, reaction of a corresponding compound of formula I in which there is a —C(O)H group present (i.e. R¹ and/or R² represents —C(O)H), with a compound of formula VIII,

R^{xx}MgX¹ VIII

wherein R^{xx} represents C₁₋₁₁ alkyl optionally substituted by one or more substituents selected from e.g. E³ and ═O (but is preferably unsubstituted) and X¹ represents halo (e.g. iodo, bromo or, preferably, chloro), under standard Grignard reaction conditions, e.g. in the presence of an inert atmosphere and, optionally, an anhydrous solvent;
(viii) compounds of formula I in which A₁ and A₄ both represent N, A₅ represents C(R²) and A₄ₐ represents C(R^{1b}) may be prepared by reaction of a compound of formula IX,

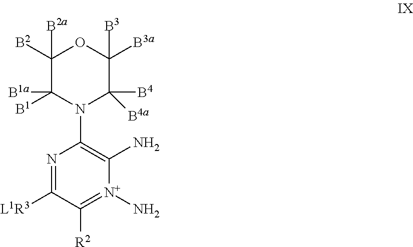

wherein L¹R³ represents either L¹ as hereinbefore defined or R³ as hereinbefore defined, and R², B¹, B^{1a}, B², B^{2a}, B³, B^{3a}, B⁴ and B^{4a} are as hereinbefore defined, with a compound of formula X,

H—C(O)—R^{1b} X

wherein R^{1b} represents —C(═Y)N(R^{10a})R^{11a} or —C(═Y)R^{10a}, optionally in the presence of a suitable base (for instance a sterically hindered base, such as an amidine base, e.g. DBU) and a suitable solvent (e.g. dichloromethane) at an appropriate temperature (e.g. room temperature) for an appropriate period of time. When L¹R³ in the compound of formula IX represents L¹, then this process step may be proceeded by process step (i) as defined above. Corresponding reactions may also take place in which A₅ represents N (instead of C(R²));
(ix) compounds of formula I in which A₁ represents N, A₄ represents C(R^{1a}). A₄ₐ represents N and A₅ represents C(R²) may be prepared by reaction of a compound of formula XI,

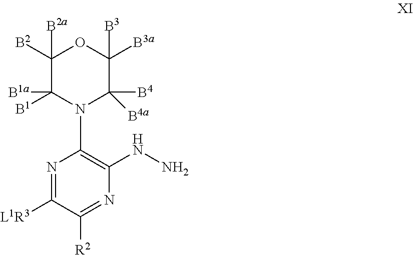

wherein L¹R³, R², B¹, B^{1a}, B², B^{2a}, B³, B^{3a}, B⁴ and B^{4a} are as hereinbefore defined, with a compound of formula XII,

R^{1a}—C(OC₁₋₆alkyl)₃ XII

or, a compound of formula XIII,

R^{1a}—C(O)OH XIII

or, derivatives of either, wherein R^{1a} represents —C(═Y)N(R^{10a})R^{11a} or —C(═Y) R^{10a} as hereinbefore defined (and is preferably hydrogen or optionally substituted C₁₋₁₂ alkyl; so forming e.g. triethyl orthoformate, triethyl orthoacetate, formic acid, and the like), under standard reaction conditions. When L¹R³ in the compound of formula XI represents L¹, then this process step may be proceeded by process step (i) as defined above. Corresponding reactions may also take place in which A₅ represents N (instead of C(R²));

(x) for compounds of formula I that contain an unsubstituted amino group (e.g. a R^{2a} or R^{2b} substituent that represents —N(R^{5a})R^{5b} in which R^{5a} and R^{5b} represent hydrogen), may be prepared by reaction of a corresponding amino protected compound of formula I (e.g. in which the —NH₂ moiety is substituted by two 4-methoxybenzyl moieties), for instance under standard reaction conditions that may be specific to that particular protecting group (e.g. by reaction in the presence of acid, such as a mixture of trifluoroacetic acid and sulfuric acid);

(xi) for compounds of formula I that contain an amino group attached to an aromatic group (e.g. a —N(R^{5a})R^{5b} moiety), reaction of a compound corresponding to a compound of formula but in which there is a halo (e.g. bromo, iodo or, preferably, fluoro) group in that position by reaction of an amine (e.g. a compound of formula HN(R^{5a})R^{5b}, or a protected derivative thereof) under appropriate coupling conditions known to those skilled in the art such as those conditions described hereinbefore in respect of process step (ii) above, for instance in the presence of DIPEA in an anhydrous solvent (e.g. dry dioxane).

Compounds of formula II may be prepared by reaction of a compound of formula XIV,

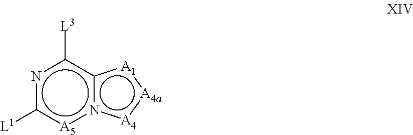

wherein L¹, L³, A₁, A₄, A₄ₐ, A₅ and R³ are as hereinbefore defined, with a compound of formula V, as hereinbefore defined, for example under reaction conditions such as those hereinbefore described in respect of preparation of compounds of formula I (process step (ii) above).

Compounds of formula IV (for example, in which A₁ represents C(R¹), A₄ represents C(R^{1a}), A₄ₐ represents C(R^{1b}) and A₅ represents C(R²)) in which L³ represents e.g. chloro, bromo or iodo, may be prepared by reaction of a compound of formula XV,

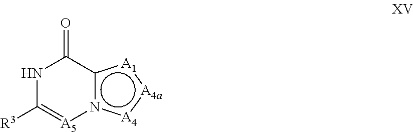

wherein A₁, A₄, A₄ₐ, A₅ and R³ as hereinbefore defined (or its tautomer), in the presence of a suitable reagent that provides the source of the chloro, bromo or iodo (e.g. POCl₃ may be employed, or, a reagent such as p-toluenesulfonyl chloride or the like) under reaction conditions known to the skilled person, for example at reflux (e.g. in the case of reaction with POCl₃) or, in the case of reaction with p-toluenesulfonyl chloride, in the presence of a base, such as an organic amine e.g. triethylamine, N,N-dimethylaniline (or the like), and optionally a catalyst such as DMAP (and optionally in the presence of a suitable solvent, such as acetonitrile). In the case of the latter, the compound of formula I may be prepared directly form the intermediate compound IV that may be formed by reaction in the presence of a compound of formula V (which latter reaction need not follow the reaction conditions set out above in respect of process step (ii); for instance, the reaction mixture may simply be heated in the same pot, e.g. at elevated temperature such as at about 65° C.

Compounds of formula IV in which L³ represents a sulfonate, such as —S(O)₂C₁₋₆ alkyl (e.g.—S(O)₂CH₃) may be prepared by oxidation of a compound of formula XVI,

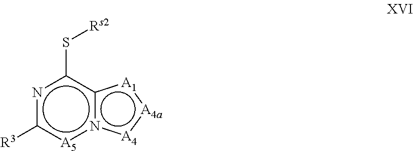

wherein R^{s2} represents C₁₋₆ alkyl (e.g. methyl), and A₁, A₄, A₄ₐ, A₅ and R³ are as hereinbefore defined, in the presence of an oxidising agent such as m-chloroperbenzoic acid and, if necessary, a suitable solvent (e.g. dichloromethane).

Compounds of formula VIA in which A₄ₐ represents C(R^{1b}) in which R^{1b} represents —C(O)OR^{10a} (and A₄ represents C(R^{1a})), may be prepared by reaction of a compound of formula XVIA,

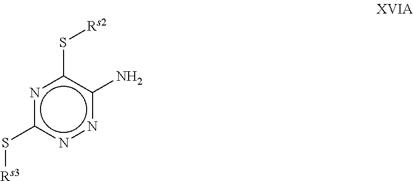

wherein R^{s3} represents C₁₋₆ alkyl (preferably methyl), with a compound of formula XVIB,

L¹⁵-C(H)(R^{1a})—C(O)—C(O)OR^{10a} XVIB

wherein L¹⁵ represents a suitable leaving group, such as one hereinbefore defined by L¹ (e.g. halo, such as bromo), and R^{1a} and R^{10a} are as hereinbefore defined (but R^{10a} is preferably not hydrogen; e.g. it represents alkyl, such as ethyl), for instance the compound of formula XVIB may be ethyl bromopyruvate, under reaction conditions known to those skilled in the art, for instance in the presence of an acid (e.g. an organic acid such as para-toluenesulfonic acid) in the presence of a suitable solvent (e.g. an aromatic solvent such as toluene), which reaction mixture may be heated at elevated temperature, e.g. at reflux. This reaction may be followed by (if and as appropriate) conversion of the relevant —S—R³ moieties to other suitable leaving groups (e.g. by oxidation to —S(O)₂—R³) and/or reaction with a compound of formula V or III as hereinbefore defined (and as appropriate). Alternatively, the reaction of the compound of formula XVIB defined above may take place with a compound of formula XVIA but in which the respective —S—R³ groups are replaced with a -L¹-R³ and an optionally substituted morpholinyl moiety, as appropriate.

Compounds of formula IX (e.g. in which L¹R³ represents L¹) may be prepared by reaction of a compound of formula XVII,

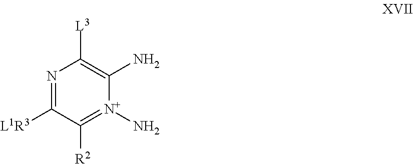

wherein L¹R³, R² and L³ are as hereinbefore defined, with a compound of formula V as hereinbefore defined, for example under reaction conditions such as those hereinbefore defined in respect of preparation of compounds of formula I (process step (ii) above).

Compounds of formula IX and XVII may be prepared by reaction of a compound of formula XVIII,

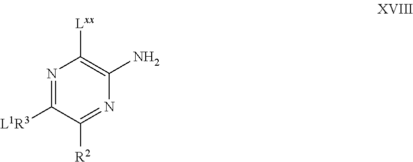

wherein L^{xx} represents L³ (in the case of preparation of compounds of formula XVII) or represents the following moiety:

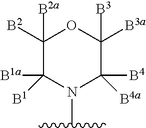

(in the case of preparation of compounds of formula IX), and R², L¹R³, B¹, B^{1a}, B², B^{2a}, B³, B^{3a}, B⁴ and B^{4a} are as hereinbefore defined, with o-(mesitylsulfonyl)hydroxylamine (or the like; i.e. another suitable source of —NH₂), under standard reaction conditions known to those skilled in the art, e.g. in the presence of a suitable solvent (e.g. dichloromethane).

Compounds of formula XI may be prepared by reaction of a compound of formula XIX,

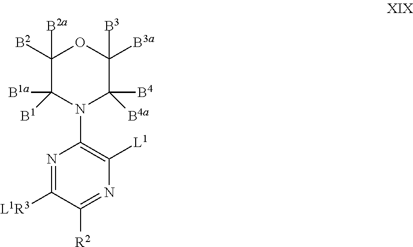

wherein L¹, R², L¹R³, B¹, B^{1a}, B², B^{2a}, B³, B^{3a}, B⁴ and B^{4a} are as hereinbefore defined, with hydrazine (or a derivative thereof, e.g. hydrazine hydrate), under standard conditions.

Compounds of formula XIV in which L¹ represents a sulfide such as —SCH₃, L³ represents a sulfide such as —SCH₃, and A₁ and A₅ both represent N, A₄ represents C(R^{1a}) and A₄ₐ represents C(R^{1b}) may be prepared by reaction of a compound of formula XX,

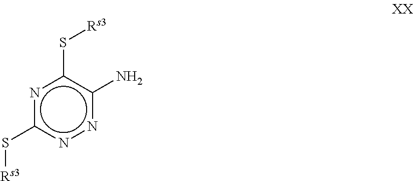

wherein R^{s3} represents C₁₋₆, alkyl (preferably methyl), with a compound of formula XXI,

L¹⁵-C(H)(R^{1a})—C(O)—R^{1b} XXI

wherein L¹⁵ represents a suitable leaving group, such as one hereinbefore defiend by L¹ (e.g. halo, such as bromo) and R^{1a} and R^{1b} are as hereinbefore defined, and R^{1a} preferably represents hydrogen (or a protected derivative thereof; e.g. the compound of formula XXI may be bromoacetaldehyde diethyl acetal, or, when R^{1b} represents —C(O)Oethyl, the compound of formula XXI may be ethyl bromopyruvate), for example in the presence of an acid catalyst (e.g. p-toluenesulfonic acid or the like), which reaction may be performed at room temperature or preferably at elevated temperature e.g. at about 65° C. Corresponding reactions may also take place in which A₅ represents C(R²).

Compounds of formula XIV in which L³ represents halo (e.g. chloro) and L¹ represents a sulfide (e.g. —SCH₃) (and, preferably, A₅ represents N, A₄ represents C(R^{1a}), A₄ₐ represents C(R^{1b}) and A₁ represents N), may also be prepared by reaction of a compound of formula XXII,

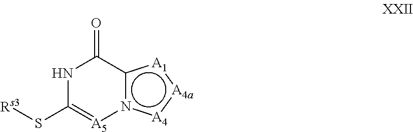

wherein A₁, A₄, A₄ₐ, A₅ and R^{s3} are as hereinbefore defined (and R^{s3} represents a group defined by R^{s2} and is preferably methyl), under halogenation reaction conditions such as those described herein, e.g. in the presence POCl₃.

Compounds of formula XIV in which L³ represents halo (especially chloro) may be prepared by reaction of a compound of formula XXIII,

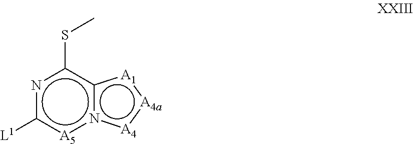

wherein L¹, A₁, A₄, A₄ₐ and A₅ are as hereinbefore defined, for example, in the presence of a base such as a metal hydroxide (e.g. KOH), in the presence of solvent (e.g. an alcohol such as methanol), followed by isolation of any intermediate product and then reaction under conditions such as those hereinbefore described in respect of preparation of compounds of formula IV (e.g. the conditions deployed in the reaction of a compound of formula XV in the presence of POCl₃, which reaction mixture may be heated at reflux for an appropriate period of time).

Compounds of formula XV may be prepared by reaction of a corresponding compound of formula XXIV,

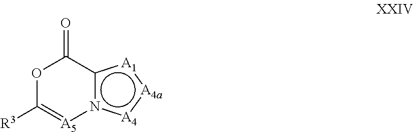

wherein A₁, A₄, A₄ₐ, A₅ and R³ as hereinbefore defined, in the presence of a suitable reagent for the replacement of the —O— moiety with a —N(H)— moiety, for example ammonia or a source thereof (e.g. ammonium acetate), under standard reaction conditions, for instance optionally in the presence of a suitable solvent (e.g. acetic acid), at elevated temperature (e.g. at about 160° C. under microwave irradiation reaction conditions).

Compounds of formula XV in which A₅ represents C(R²) (and preferably, A₁ represents C(R¹), A₄ represents N, A₄ₐ represents C(R^{1b}) and A₅ represents C(H); further, R^{1b} may represent —C(O)OR^{10a}) may also be prepared by reaction of a compound of formula XXV,

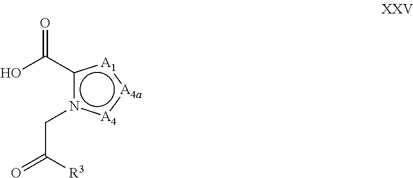

or a derivative thereof (e.g. a carboxylic acid ester such as a —C(O)O-ethyl, for instance A₄ₐ may represent —C(R^{1b}), in which R^{1b} represents —C(O)OR^{10a} and R^{10a} is preferably ethyl), wherein A₁, A₄, A₄ₐ and R³ are as hereinbefore defined (but, preferably, A₁ represents C(R¹), A₄ represents N and A₄ₐ represents C(R^{1b}), in which R^{1b} may represent —C(O)OR^{10a}), with a source of ammonia, such as ammonium acetate, for example under reaction conditions such as those described herein (e.g. above), or in the presence of an alcoholic solvent (e.g. ethanol).

Compounds of formula XV, or protected derivatives thereof (which includes salts, e.g. a bromide salt), in which A₅ represents C(R²) (and, preferably, A₄ₐ represents N and/or, preferably, A₁ represents C(R¹) and A₄ represents C(R^{1a})) may be prepared by reaction of a compound of formula XXVI,

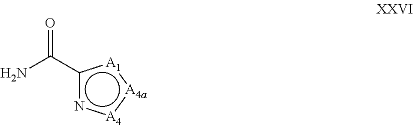

or a protected derivative thereof, e.g. a methyl protected derivative thereof, for instance, when the A₁ to A₄ₐ-containing ring represents an imidazole ring (i.e. A₄ₐ represents N, and the other ring members are C, then the N at A₄ₐ may be protected, e.g. by a methyl group, so forming for example 1-methyl-1H-imidazole-4-carboxamide) and wherein A₁, A₄ and A₄ₐ are as hereinbefore defined, with a compound of formula XXVII,

L¹²-C(H)(R²)—C(O)—R³ XXVII

wherein L¹² represents a suitable leaving group, such as one hereinbefore defined in respect of L¹ (e.g. halo, preferably, bromo), and R² and R³ are as hereinbefore defined (and R² is preferably hydrogen), for example at elevated temperature (e.g at reflux) in the presence of an appropriate solvent (e.g. acetonitrile, dimethylformamide, and the like, or mixtures thereof).

Compounds of formula XV, in which A₅ represents N (e.g. A₁, Ag and A₄ₐ respectively represent C(R¹), C(R^{1a}) and C(R^{1b}) and A₅ represents N, or, A₁ represents N(R^{1x}), A₄ represents C(R^{1a}) and A₄ₐ and A₅ both represent N) may also be prepared by intramolecular cyclisation of a compound of formula XXVIII,

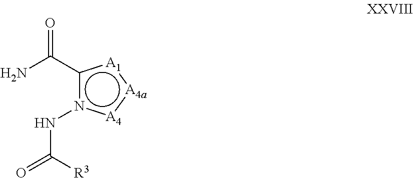

wherein R³, A₁, A₄ and A₄ₐ are as hereinbefore defined, by reaction in the presence of a base, for instance an aqueous basic solution such as ammonium hydroxide, or a metal alkyl-oxide (e.g. potassium tert-butoxide) in an alcoholic solution (e.g. butanol), for instance at elevated temperature e.g. at about 120° C. under microwave irradiation reaction conditions.

Compounds of formula XV, in which R³ is replaced with a —OH group and A₅ represents N (e.g. A₁, A₄ and A₄ₐ respectively represent C(R¹), C(R^{1a}) and C(R^{1b}) and A₅ represents N, or, A₁ represents N(R^{1x}), A₄ represents C(R^{1a}) and A₄ₐ and A₅ both represent N) may also be prepared by reaction of a compound of formula XXIX,

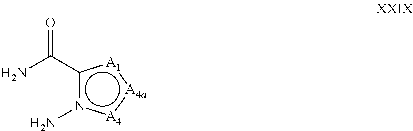

wherein A₁, A₄ and A₄ₐ are as hereinbefore defined, with phosgene, triphosgene, carbonyl diimidazole, or the like, i.e. another suitable reagent that acts as a similar source of a carbonyl group, under reaction conditions such as those described hereinafter. Such amido-compounds may be prepared by coupling of the corresponding carboxylic acid with ammonia (or a suitable source thereof, e.g. NH₄Cl in NH₃/MeOH).

Compounds of formula XVI may be prepared by reaction of a compound of formula XXX,

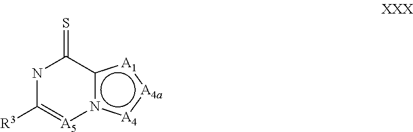

wherein A₁, A₄, A₄ₐ, A₅ and R³ are as hereinbefore defined, with a compound of formula XXXI,

R^{s2}-L¹³ XXXI

wherein L¹³ represents a suitable leaving group (such as halo, e.g. iodo) and R^{s2} is as hereinbefore defined (e.g. methyl iodide), for example in the presence of aqueous NaOH solution and an alocoholic solvent (e.g. methanol).

Compounds of formula XVI (or the bicyclic core of compounds of formula XVI) may also be prepared by intramolecular reaction of a compound of formula XXXII,

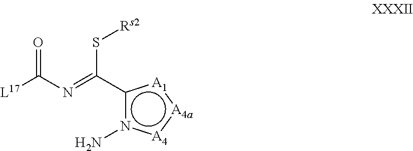

wherein L¹⁷ represents a suitable leaving group (e.g. halo, such as chloro), A₁ is preferably N, A₄ is C(R^{1a}) and A₅ is C(R^{1b}), for example in the presence of base at elevated temperature, followed by standard modification and/or introduction of functional groups.

Compounds of formula XVIA may be prepared by reaction of a compound of formula XXXIIA,

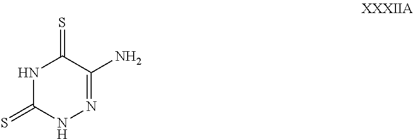

with a compound of formula XXXIIB,

L¹⁸-R^{s3} XXXIIB

wherein L¹⁸ represents a suitable leaving group, such as one hereinbefore defined by L¹ (and preferably bromo, chloro or especially iodo), for instance in the presence of a base (e.g. an organic amine, such as DIPEA or the like) and a suitable solvent (e.g. DCM).

Compounds of formula XVII may be prepared by reaction of a compound of formula XXXIII,

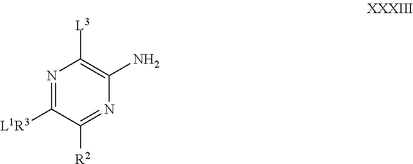

wherein R², L¹R³ and L³ are as hereinbefore defined, with a suitable aminating agent, for instance a hydroxylamine compound (e.g. a sulfonyl-hydroxylamine, such as o-(meistylsulfonyl)hydroxylamine), under standard reaction conditions.

Compounds of formula XIX in which L¹ represents chloro (or halo) may be prepared by reaction of a compound of formula XXXIV,

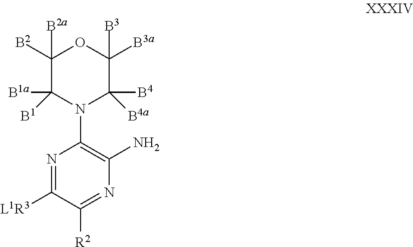

wherein L¹R³, R², B¹, B^{1a}, B², B^{2a}, B³, B^{3a}, B⁴ and B^{4a} are as hereinbefore defined, with a reagent, or mixture of reagents, that are suitable for converting the amino moiety to a chloro (or other halo) moiety, for example, TiCl₄ and tert-butyl nitrite, under conditions such as those described hereinafter.

Compounds of formula XXII in which A₅ represents N (and preferably in which A₅ represents N, A₄ represents C(R^{1a}), A₄ₐ represents C(R^{1b}) and A₁ represents N) may be prepared by reaction of a compound of formula XXXV,

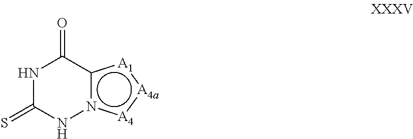

wherein A₁, A₄ and A₄ₐ are as hereinbefore defined (but, preferably, A₄ represents C(R^{1a}), A₄ₐ represents C(R^{1b}) and A₁ represents N), in the presence of a compound of formula XXXI as hereinbefore defined but in which R^{s2} represents R^{s3}.

Compounds of formula XXIV in which in which A₅ represents C(R²) (and, preferably, A₁ represents C(R¹), A₄ represents C(R^{1a}), A₄ₐ represents C(R^{1b}) and A₅ represents C(H)), may be prepared by reaction of a compound of formula XXXVI,

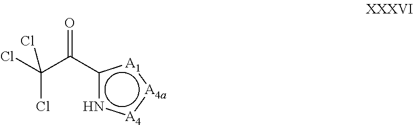

wherein A₁, A₄ and A₄ₐ are as hereinbefore defined, with a compound of formula XXXVII,

R³—C(O)—CH₂-L⁹ XXXVII

wherein L⁹ represents a suitable leaving group, for example one hereinbefore defined in respect of L¹ (e.g. halo, and preferably, bromo), under standard reaction conditions, for example, optionally in the presence of a suitable base (preferably an inorganic base, such as NaH, K₃PO₄, Cs₂CO₃, t-BuONa, t-BuOK, and, more preferably an inorganic carbonate such as Na₂CO₃ and, preferably, K₂CO₃) and a suitable solvent (e.g. an aprotic solvent such as dichloromethane or, preferably, acetone). The reaction may be performed at elevated temperature, for example, at above 100° C. (e.g. at about 120° C.) under microwave irradiation conditions.

Compounds of formula XXV may be prepared by reaction of a compound of formula XXXVIII,

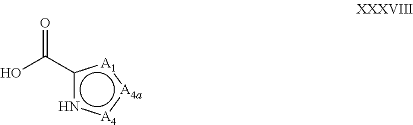

or a derivative thereof (e.g. ester such as ethyl ester), wherein A₁, A₄ and A₄ₐ are as hereinbefore defined, with a compound of formula XXXIX,

L¹⁰-CH₂—C(O)—R³ XXXIX

wherein L¹⁰ represents a suitable leaving group, such as one hereinbefore defined in respect of L¹ (e.g. halo, such as bromo), and R³ is as hereinbefore defined, under standard reaction conditions, for example optionally in the presence of a suitable base and solvent (such as those hereinbefore described in respect of preparation of compounds of formula XXIV (by reaction of a compound of formula XXXVI and XXXVII), e.g. K₂CO₃ in acetone).
Compounds of formula XXVI may be prepared by reaction of a compound of formula XXXVIII as hereinbefore defined, or a derivative thereof (e.g. an ester, such as an ethyl ester), with ammonia or a suitable source thereof (e.g. NH₄Cl in a solution of NH₃ in an alcohol such as methanol).

Compounds of formula XXVIII may be prepared by reaction of a compound of formula XXIX as hereinbefore defined with a compound of formula XL,

R³—C(O)-L¹¹ XL

wherein L¹¹ represents a suitable leaving group such as one hereinbefore defined by L¹ (e.g. halo, such as chloro) or —OH (or an ester, thereof) under standard acylation or amide coupling reaction conditions, e.g. in the case of acylation, the presence of an appropriate base (e.g. an organic amine base such as triethylamine) and an appropriate solvent (e.g. pyridine, dichloromethane, dioxane, etc, or mixtures thereof), or, in the case of amide couplings, under conditions described hereinafter (or e.g. in the presence of polyphosphoric acid, which advantageously may form a compound of formula XXVIII in situ, which may undergo subsequent reaction to provide the compound of formula XV isoquinolinone directly).

Compounds of formula XXIX may be prepared by (partial) hydrolysis of a compound of formula XLI,

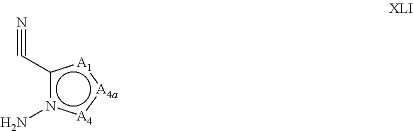

wherein A₁, A₄ and A₄ₐ are as hereinbefore defined, under standard hydrolysis reaction conditions, e.g. in the presence of an aqueous hydroxide base (e.g. potassium hydroxide) in a suitable solvent such as tetrahydrofuran.

Compounds of formula XXIX (or the corresponding carboxylic acid or ester) may also be prepared by amination of a compound of formula XXVI as hereinbefore defined, or compounds of formula XLI may also be prepared by amination of a compound of formula XLII,

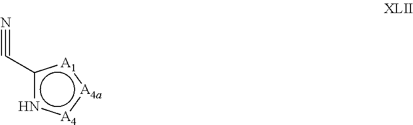

wherein A₁, A₄ and A₄ₐ are as hereinbefore defined, under reaction conditions such as those described hereinafter, e.g. in the presence of sodium hydride, followed by o-(diphenylphosphinyl)hydroxylamine.

Compounds of formula XXXIIA may be prepared by reaction of a compound of formula XLIIA,

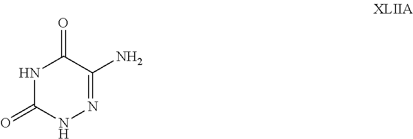

In the presence of a suitable reagent for the conversion of a carbonyl group to a thiocarbonyl group, for instance in the presence of Lawesson's reagent or phosphorous pentasulfide, in the presence of a suitable solvent (e.g. dry pyridine), which reaction mixture may be heated at elevated temperature, for instance at reflux.

Compounds of formula XXXIX in which L¹⁰ represents halo (e.g. chloro or, preferably, bromo) may be prepared by reaction of a compound corresponding to a compound of formula XXXIX but in which L¹⁰ represents hydrogen, with a source of halide ions (e.g. such as one hereinbefore described in respect of preparation of compounds of formula I; process step (iii) above), such as N-chlorosuccinimide or N-bromosuccinimide, under standard reaction conditions e.g. in the presence of a suitable base (such as an organic base e.g. triethylamine or the like) and trimethylsilylfluoromethanesulfonate, or the like.

Compounds corresponding to compounds of formula XXXIX but in which L¹⁰ represents hydrogen may themselves be prepared from compounds of formula XLIII,

R³-L¹¹ XLIII

in which L¹¹ represents a suitable leaving group, such as one hereinbefore defined in respect of L¹ (e.g. halo, such as chloro or, preferably bromo), with a compound that allows the introduction of the —C(O)CH₃ moiety, such as tributyl(1-ethoxyvinyl)tin in the presence of a precious metal catalyst/ligand (e.g. dichlorobis(triphenyl-phosphine)palladium (II)) and a suitable solvent (e.g. dimethylformamide, or the like).

Compounds of formula XLI may be prepared by reaction of a compound of formula XLII as hereinbefore defined, for example by reaction in the presence of base (e.g. a metal hydride, such as sodium hydride) and an appropriate reagent for the introduction of the amino group, e.g. o-(diphenylphosphinyl)-hydroxylamine, or another reagent suitable for electrophilic aminations, under reaction conditions such as those described hereinafter.

Compounds of formula XLII may be prepared by reaction of a compound of formula XLIV,

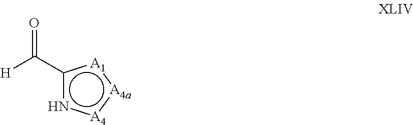

wherein A₁, A₄ and A₄ₐ are as hereinbefore defined, in the presence of hydroxylamine (e.g. the hydrochloride thereof), followed by dehydration (in the presence of a suitable dehydrating agent, such as phthalic anhydride).

Compounds of formula XLIIA may be prepared by reaction of a corresponding compound of formula XLIVA,

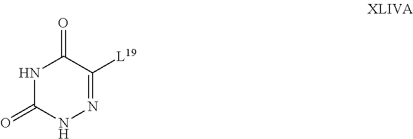

wherein L¹⁹ represents a suitable leaving group such as one hereinbefore defined by L¹ (e.g. bromo), in the presence of a source of ammonia (e.g. liquid ammonia), for instance under coupling reaction conditions e.g. in the presence of a metal catalyst e.g. copper.

Compounds of formula XLIVA may be prepared using standard methods, for instance, by the reaction of 6-azauracil in the presence of bromine and water.

Other specific transformation steps (including those that may be employed in order to form compounds of formula I) that may be mentioned include:

(i) reductions, for example of a carboxylic acid (or ester) to either an aldehyde or an alcohol, using appropriate reducing conditions (e.g. —C(O)OH (or an ester thereof), may be converted to a —C(O)H or —CH₂—OH group, using DIBAL and LiAlH₄, respectively (or similar chemoselective reducing agents));
(ii) reductions of an aldehyde (—C(O)H) group to an alcohol group (—CH₂OH), using appropriate reduction conditions such as those mentioned at point (i) above;
(iii) oxidations, for example of a moiety containing an alcohol group (e.g. —CH₂OH) to an aldehyde (e.g. —C(O)H), for example in the presence of a suitable oxidising agent, e.g. MnO₂ or the like;
(iv) reductive amination of an aldehyde and an amine, under appropriate reaction conditions, for example in “one-pot” procedure in the presence of an appropriate reducing agent, such as a chemoselective reducing agent such as sodium cyanoborohydride or, preferably, sodium triacetoxyborohydride, or the like. Alternatively, such reactions may be performed in two steps, for example a condensation step (in the presence of e.g. a dehydrating agent such as trimethyl orthoformate or MgSO₄ or molecular sieves, etc) followed by a reduction step (e.g. by reaction in the presence of a reducing agent such as a chemoselective one mentioned above or NaBH₄, AIH₄, or the like);
(v) amide coupling reactions, i.e. the formation of an amide from a carboxylic acid (or ester thereof), for example when R² represents —C(O)OH (or an ester thereof), it may be converted to a —C(O)N(R^{10b})R^{11b} group (in which R^{10b} and R^{11b} are as hereinbefore defined, and may be linked together, e.g. as defined above), and which reaction may (e.g. when R² represents —C(O)OH) be performed in the presence of a suitable coupling reagent (e.g. 1,1′-carbonyldiimidazole, N,N′-dicyclohexylcarbodiimide, or the like) or, in the case when R² represents an ester (e.g. —C(O)OCH₃ or —C(O)OCH₂CH₃), in the presence of e.g. trimethylaluminium, or, alternatively the —C(O)OH group may first be activated to the corresponding acyl halide (e.g —C(O)Cl, by treatment with oxalyl chloride, thionyl chloride, phosphorous pentachloride, phosphorous oxychloride, or the like), and, in all cases, the relevant compound is reacted with a compound of formula HN(R^{10a})R^{11a} (in which R^{10a} and R^{11a} are as hereinbefore defined), under standard conditions known to those skilled in the art (e.g. optionally in the presence of a suitable solvent, suitable base and/or in an inert atmosphere);
(vi) conversion of a primary amide to a nitrile functional group, for example under dehydration reaction conditions, e.g. in the presence of POCl₃, or the like;
(vii) nucleophilic substitution reactions, where any nucleophile replaces a leaving group, e.g. methylsulfonylpiperazine may replace a chloro leaving group, or, aromatic nucleophilic substitution reactions such as the substitution of ammonia (or a protected derivative thereof, e.g. a dibenzyl derivative) onto an aromatic group bearing a leaving group (e.g. onto a 2-chloropyrimidinyl moiety);
(viii) transformation of a methoxy group to a hydroxy group, by reaction in the presence of an appropriate reagent, such as boron fluoride-dimethyl sulfide complex or BBr₃ (e.g. in the presence of a suitable solvent such as dichloromethane);
(ix) specific deprotection steps, for example a hydroxy group protected as a silyl ether (e.g. a tert-butyl-dimethylsilyl protecting group) may be deprotected by reaction with a source of fluoride ions, e.g. by employing the reagent tetrabutylammonium fluoride (TBAF).

Intermediate compounds described herein are either commercially available, are known in the literature, or may be obtained either by analogy with the processes described herein, or by conventional synthetic procedures, in accordance with standard techniques, from available starting materials using appropriate reagents and reaction conditions. Further, processes to prepare compounds of formula I may be described in the literature, for example in:
Werber, G. et al.; J. Heterocycl. Chem.; EN; 14; 1977; 823-827;
Andanappa K. Gadad et al. Bioorg. Med. Chem. 2004, 12, 5651-5659;
Paul Heinz et al. Monatshefte für Chemie, 1977, 108, 665-680;
M. A. El-Sherbeny et al. Boll. Chim. Farm. 1997, 136, 253-256;
Nicolaou, K. C.; Bulger, P. G.; Sarlah, D. Angew. Chem. Int. Ed. 2005, 44, 2-49;
Bretonnet et al. J. Med. Chem. 2007, 50, 1872;
Asunción Marin et al. Farmaco 1992, 47 (1), 63-75;
Severinsen, R. et al. Tetrahedron 2005, 61, 5565-5575;
Nicolaou, K. C.; Bulger, P. G.; Sarlah, D. Angew. Chem. Int. Ed. 2005, 44, 2-49;
M. Kuwahara et al., Chem. Pharm Bull., 1996, 44, 122;
Wipf, P.; Jung, J.-K. J. Org. Chem. 2000, 65(20), 6319-6337;
Shintani, R.; Okamoto, K. Org. Lett. 2005, 7 (21), 4757-4759;
Nicolaou, K. C.; Bulger, P. G.; Sarlah, D. Angew. Chem. Int. Ed. 2005, 44, 2-49;
J. Kobe et al., Tetrahedron, 1968, 24, 239;
P. F. Fabio, A. F. Lanzilotti and S. A. Lang, Journal of Labelled Compounds and Pharmaceuticals, 1978, 15, 407;
F. D. Bellamy and K. Ou, Tetrahedron Lett., 1985, 25, 839;
M. Kuwahara et al., Chem. Pharm Bull., 1996, 44, 122;
A. F. Abdel-Magid and C. A Maryanoff. Synthesis, 1990, 537;
M. Schlosser et al. Organometallics in Synthesis. A Manual, (M. Schlosser, Ed.),
Wiley &Sons Ltd: Chichester, UK, 2002, and references cited therein;
L. Wengwei et al., Tetrahedron Lett., 2006, 47, 1941;
M. Plotkin et al. Tetrahedron Lett., 2000, 41, 2269;
Seyden-Penne, J. Reductions by the Alumino and Borohydrides, VCH, NY, 1991;
O. C. Dermer, Chem. Rev., 1934, 14, 385;
N. Defacqz, et al., Tetrahedron Lett., 2003, 44, 9111;
S. J. Gregson et al., J. Med. Chem., 2004, 47, 1161;
A. M. Abdel Magib, et al., J. Org. Chem., 1996, 61, 3849;
A. F. Abdel-Magid and C. A Maryanoff. Synthesis, 1990, 537;
T. Ikemoto and M. Wakimasu, Heterocycles, 2001, 55, 99;
E. Abignente et al., II Farmaco, 1990, 45, 1075;
T. Ikemoto et al., Tetrahedron, 2000, 56, 7915;
T. W. Greene and P. G. M. Wuts, Protective Groups in Organic Synthesis, Wiley, NY, 1999;
S. Y. Han and Y.-A. Kim. Tetrahedron, 2004, 60, 2447;
J. A. H. Lainton et al., J. Comb. Chem., 2003, 5, 400; or
Wiggins, J. M. Synth. Commun., 1988, 18, 741.

The substituents R³, B¹, B^{1a}, B², B^{2a}, B³, B^{3a}, B⁴, B^{4a}, A₁, A A₂, A₃, A₄, A₄ₐ and A₅ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence.

For example, when substituents in the compounds of the invention (e.g. represented by R³, B¹, B^{1a}, B², B^{2a}, B³, B^{3a}, B⁴, B^{4a}, A₁, A₂, A₃, A₄, A₄ₐ and A₅) such as CO₂Et, CHO, CN and/or CH₂Cl, are present, these groups can be further derivatized to other fragments described (e.g. by those integers mentioned above) in compounds of the invention, following synthetic protocols very well know to the person skilled in the art and/or according to the experimental part described in the patent. Other specific transformation steps that may be mentioned include: the reduction of a nitro or azido group to an amino group; the hydrolysis of a nitrile group to a carboxylic acid group; and standard nucleophilic aromatic substitution reactions, for example in which an iodo-, preferably, fluoro- or bromo-phenyl group is converted into a cyanophenyl group by employing a source of cyanide ions (e.g. by reaction with a compound which is a source of cyano anions, e.g. sodium, copper (I), zinc or potassium cyanide, optionally in the presence of a palladium catalyst) as a reagent (alternatively, in this case, palladium catalysed cyanation reaction conditions may also be employed).

Other transformations that may be mentioned include: the conversion of a halo group (preferably iodo or bromo) to a 1-alkynyl group (e.g. by reaction with a 1-alkyne), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate nucleophile e.g. a source of the relevant anions, for example by reaction in the presence of a halogen gas (e.g. bromine, iodine or chlorine), or a reagent that is a source of azido or cyanide anions, such as NaN₃ or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Compounds of the invention bearing a carboxyester functional group may be converted into a variety of derivatives according to methods well known in the art to convert carboxyester groups into carboxamides, N-substituted carboxamides, N,N-disubstituted carboxamides, carboxylic acids, and the like. The operative conditions are those widely known in the art and may comprise, for instance in the conversion of a carboxyester group into a carboxamide group, the reaction with ammonia or ammonium hydroxide in the presence of a suitable solvent such as a lower alcohol, dimethylformamide or a mixture thereof; preferably the reaction is carried out with ammonium hydroxide in a methanol/dimethyl-formamide mixture, at a temperature ranging from about 50° C. to about 100° C. Analogous operative conditions apply in the preparation of N-substituted or N,N-disubstituted carboxamides wherein a suitable primary or secondary amine is used in place of ammonia or ammonium hydroxide. Likewise, carboxyester groups may be converted into carboxylic acid derivatives through basic or acidic hydrolysis conditions, widely known in the art. Further, amino derivatives of compounds of the invention may easily be converted into the corresponding carbamate, carboxamido or ureido derivatives.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The need for such protection will vary depending on the nature of the remote functionality and the conditions of the preparation methods (and the need can be readily determined by one skilled in the art). Suitable amino-protecting groups include acetyl, trifluoroacetyl, t-butoxycarbonyl (BOC), benzyloxycarbonyl (CBz), 9-fluorenylmethyleneoxycarbonyl (Fmoc) and 2,4,4-trimethylpentan-2-yl (which may be deprotected by reaction in the presence of an acid, e.g. HCl in water/alcohol (e.g. MeOH)) or the like. The need for such protection is readily determined by one skilled in the art.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P.G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined, for use as a pharmaceutical.

Compounds of the invention may inhibit protein or lipid kinases, such as a PI3 kinase (especially a class I PI3K), for example as may be shown in the tests described below (for example, the test for PI3Kα inhibition described below) and/or in tests known to the skilled person. The compounds of the invention may also inhibit mTOR. Thus, the compounds of the invention may be useful in the treatment of those disorders in an individual in which the inhibition of such protein or lipid kinases (e.g. PI3K, particularly class I PI3K, and/or mTOR) is desired and/or required (for instance compounds of the invention may inhibit PI3K, particularly class I PI3K and, optionally, may also inhibit mTOR).

The term “inhibit” may refer to any measurable reduction and/or prevention of catalytic kinase (e.g. PI3K, particularly class I PI3K, and/or mTOR) activity. The reduction and/or prevention of kinase activity may be measured by comparing the kinase activity in a sample containing a compound of the invention and an equivalent sample of kinase (e.g. PI3K, particularly class I PI3K, and/or mTOR) in the absence of a compound of the invention, as would be apparent to those skilled in the art. The measurable change may be objective (e.g. measurable by some test or marker, for example in an in vitro or in vivo assay or test, such as one described hereinafter, or otherwise another suitable assay or test known to those skilled in the art) or subjective (e.g. the subject gives an indication of or feels an effect).

Compounds of the invention may be found to exhibit 50% inhibition of a protein or lipid kinase (e.g. PI3K, such as class I PI3K, and/or mTOR) at a concentration of 100 μM or below (for example at a concentration of below 50 μM, or even below 10 μM, such as below 1 μM), when tested in an assay (or other test), for example as described hereinafter, or otherwise another suitable assay or test known to the skilled person.

Compounds of the invention are thus expected to be useful in the treatment of a disorder in which a protein or lipid kinase (e.g. PI3K, such as class I PI3K, and/or mTOR) is known to play a role and which are characterised by or associated with an overall elevated activity of that kinase (due to, for example, increased amount of the kinase or increased catalytic activity of the kinase). Hence, compounds of the invention are expected to be useful in the treatment of a disease/disorder arising from abnormal cell growth, function or behaviour associated with the protein or lipid kinase (e.g. PI3K, such as class I PI3K, and/or mTOR). Such conditions/disorders include cancer, immune disorders, cardiovascular diseases, viral infections, inflammation, metabolism/endocrine function disorders and neurological disorders.

The disorders/conditions that the compounds of the invention may be useful in treating hence includes cancer (such as lymphomas, solid tumours or a cancer as described hereinafter), obstructive airways diseases, allergic diseases, inflammatory diseases (such as asthma, allergy and Chrohn's disease), immunosuppression (such as transplantation rejection and autoimmune diseases), disorders commonly connected with organ transplantation, AIDS-related diseases and other associated diseases. Other associated diseases that may be mentioned (particularly due to the key role of kinases in the regulation of cellular proliferation) include other cell proliferative disorders and/or non-malignant diseases, such as benign prostate hyperplasia, familial adenomatosis, polyposis, neuro-fibromatosis, psoriasis, bone disorders, atherosclerosis, vascular smooth cell proliferation associated with atherosclerosis, pulmonary fibrosis, arthritis glomerulonephritis and post-surgical stenosis and restenosis. Other disease states that may be mentioned include cardiovascular disease, stroke, diabetes, hepatomegaly, Alzheimer's disease, cystic fibrosis, hormone-related diseases, immunodeficiency disorders, destructive bone disorders, infectious diseases, conditions associated with cell death, thrombin-induced platelet aggregation, chronic myelogenous leukaemia, liver disease, pathologic immune conditions involving T cell activation and CNS disorders.

As stated above, the compounds of the invention may be useful in the treatment of cancer. More, specifically, the compounds of the invention may therefore be useful in the treatment of a variety of cancer including, but not limited to: carcinoma such as cancer of the bladder, breast, colon, kidney, liver, lung (including non-small cell cancer and small cell lung cancer), esophagus, gall-bladder, ovary, pancreas, stomach, cervix, thyroid, prostate, skin, squamous cell carcinoma, testis, genitourinary tract, larynx, glioblastoma, neuroblastoma, keratoacanthoma, epidermoid carcinoma, large cell carcinoma, non-small cell lung carcinoma, small cell lung carcinoma, lung adenocarcinoma, bone, adenoma, adenocarcinoma, follicular carcinoma, undifferentiated carcinoma, papilliary carcinoma, seminona, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukaemia; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xeroderma pigmentosum, keratoxanthoma, thyroid follicular cancer and Kaposi's sarcoma.

Further, the protein or lipid kinases (e.g. PI3K, such as class I PI3K, and/or mTOR) may also be implicated in the multiplication of viruses and parasites. They may also play a major role in the pathogenesis and development of neurodegenerative disorders. Hence, compounds of the invention may also be useful in the treatment of viral conditions, parasitic conditions, as well as neurodegenerative disorders.

Compounds of the invention are indicated both in the therapeutic and/or prophylactic treatment of the above-mentioned conditions.

According to a further aspect of the present invention, there is provided a method of treatment of a disease (e.g. cancer or another disease as mentioned herein) which is associated with the inhibition of protein or lipid kinase (e.g. PI3K, such as class I PI3K, and/or mTOR), i.e. where such inhibition is desired and/or required (for example, a method of treatment of a disease/disorder arising from abnormal cell growth, function or behaviour associated with protein or lipid kinases, e.g. PI3K, such as class I PI3K, and/or mTOR), which method comprises administration of a therapeutically effective amount of a compound of the invention, as hereinbefore defined, to a patient suffering from, or susceptible to, such a condition.

“Patients” include mammalian (including human) patients. Hence, the method of treatment discussed above may include the treatment of a human or animal body.

The term “effective amount” refers to an amount of a compound, which confers a therapeutic effect on the treated patient. The effect may be objective (e.g. measurable by some test or marker) or subjective (e.g. the subject gives an indication of or feels an effect).

Compounds of the invention may be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like. The type of pharmaceutical formulation may be selected with due regard to the intended route of administration and standard pharmaceutical practice. Such pharmaceutically acceptable carriers may be chemically inert to the active compounds and may have no detrimental side effects or toxicity under the conditions of use.

Such formulations may be prepared in accordance with standard and/or accepted pharmaceutical practice. Otherwise, the preparation of suitable formulations may be achieved non-inventively by the skilled person using routine techniques and/or in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent and/or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The amount of compound of the invention in the formulation will depend on the severity of the condition, and on the patient, to be treated, as well as the compound(s) which is/are employed, but may be determined non-inventively by the skilled person.

The invention further provides a process for the preparation of a pharmaceutical formulation, as hereinbefore defined, which process comprises bringing into association a compound of the invention, as hereinbefore defined, or a pharmaceutically acceptable ester, amide, solvate or salt thereof with a pharmaceutically-acceptable adjuvant, diluent or carrier.

Compounds of the invention may also be combined with other therapeutic agents that are inhibitors of protein or lipid kinases (e.g. PI3K, such as class I PI3K, a PIM family kinase (e.g. PIM-1, PIM-2- and/or PIM-3) and/or mTOR) and/or useful in the treatment of a cancer and/or a proliferative disease. Compounds of the invention may also be combined with other therapies.

According to a further aspect of the invention, there is provided a combination product comprising:
(A) a compound of the invention, as hereinbefore defined; and
(B) another therapeutic agent that is useful in the treatment of cancer and/or a proliferative disease,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of cancer and/or a proliferative disease, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of cancer and/or a proliferative disease in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable ester, amide, solvate or salt thereof with the other therapeutic agent that is useful in the treatment of cancer and/or a proliferative disease, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Depending on the disorder, and the patient, to be treated, as well as the route of administration, compounds of the invention may be administered at varying therapeutically effective doses to a patient in need thereof. However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of the invention.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

Compounds of the invention may have the advantage that they are effective inhibitors of protein or lipid kinases (e.g. PI3K, such as class I PI3K, and/or mTOR). Advantegously, compounds of the invention may inhibit (e.g. selectively) certain protein or lipid kinases (e.g. PI3K, such as class I PI3K), without exhibiting inhibition (or significant inhibition) of other protein or lipid kinases. For instance, the compounds of the invention may selectively inhibit only one protein or lipid kinase (e.g. PI3K, such as class I PI3K).

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

Compounds of the invention may be beneficial as they are medicaments with targeted therapy, i.e. which target a particular molecular entity by inferring or inhibiting it (e.g. in this case by inhibiting one or more protein or lipid kinases as hereinbefore described). Compounds of the invention may therefore also have the benefit that they have a new effect (for instance as compared to known compounds in the prior art), for instance, the new effect may be a particular mode of action or another effect resultant of the targeted therapy. Targeted therapies may be beneficial as they may have the desired effect (e.g. reduce cancer, by reducing tumor growth or carcinogenisis) but may also have the advantage of reducing side effects (e.g. by preventing the killing of normal cells, as may occur using e.g. chemotherapy).

Furthermore, compounds of the invention may selectively target particular protein or lipid kinases (e.g. the ones described herein) compared to other known protein or lipid kinases (as may be shown experimentally hereinafter; see Table 4 for example). Accordingly, compounds of the invention may have the advantage that certain, specific, cancers may be treated selectively, which selective treatment may also have the effect of reducing side effects.

### Examples/Biological Tests

Determination of the activity of PI3 kinase activity of compounds of the invention is possible by a number of direct and indirect detection methods. Certain exemplary compounds described herein were prepared, characterized, and tested for their PI3K binding activity and in vitro activity against tumor cells. The range of PI3K binding activities was less than 1 nM to about 10 μM (i.e. certain compounds of the examples/invention had PI3K binding activity IC₅₀ values of less than 10 nM). Compounds of the examples/invention had tumor cell-based activity IC₅₀ values less than 100 nM (see Table(s) below).

### PI3K Activity Assay

The kinase activity was measured by using the commercial ADP Hunter™ Plus assay available from DiscoveRₓ, (#33-016), which is a homogeneous assay to measure the accumulation of ADP, a universal product of kinase activity. The enzyme, PI3K (p110α/p85α, was purchased from Carna Biosciences (#07CBS-0402A). The assay was done following the manufacturer recommendations with slight modifications: Mainly the kinase buffer was replace by 50 mM HEPES, pH 7.5, 3 mM MgCl₂, 100 mM NaCl, 1 mM EGTA, 0.04% CHAPS, 2 mM TCEP and 0.01 mg/ml BGG. The PI3K was assayed in a titration experiment to determine the optimal protein concentration for the inhibition assay. To calculate the IC₅₀ of the ETP-compounds, serial 1:5 dilutions of the compounds were added to the enzyme at a fixed concentration (2.5 μg/ml. The enzyme was preincubated with the inhibitor and 30 μM PIP₂ substrate (P9763, Sigma) for 5 min and then ATP was added to a final 50 μM concentration. Reaction was carried out for 1 hour at 25° C. Reagent A and B were sequentially added to the wells and plates were incubated for 30 min at 37° C. Fluorescence counts were read in a Victor instrument (Perkin Elmer) with the recommended settings (544 and 580 nm as excitation and emission wavelengths, respectively). Values were normalized against the control activity included for each enzyme (i.e., 100% PI3 kinase activity, without compound). These values were plot against the inhibitor concentration and were fit to a sigmoid dose-response curve by using the Graphad software.

### Cellular Mode of Action

### Cell Culture:

The cell lines were obtained from the American Type Culture Collection (ATCC). U20S (human osteosarcoma) was cultured in Dulbecco's modified Eagle's medium (DMEM). PC3 (human prostate carcinoma), MCF7 (human breast cardinoma), HCT116 (human colon carcinoma), 768-0 (human neuroblastoma), U251 (human glyoblastoma) were grown in RPMI. All media were supplemented with 10% fetal bovine serum (FBS) (Sigma) and antibiotics-antimycotics. Cell were maintained in a humidified incubator at 37° C. with 5% CO₂ and passaged when confluent using trypsin/EDTA.

U2foxRELOC and U2nesRELOC Assay:

The U2nesRELOC assay and the U2foxRELOC assay have been described previously (1, 2). Briefly, cells were seeded at a density of 1.0×10⁵ cells/rill into black-wall clear-bottom 96-well microplates (BD Biosciences) After incubation at 37° C. with 5% CO₂ for 12 hours, 2 μl of each test compound were transferred from the mother plates to the assay plates. Cells were incubated in the presence of the compounds for one hour. Then cells were fixed and the nucleus stained with DAPI (Invitrogen). Finally the plates were washed with 1×PBS twice and stored at 4° C. before analysis. Compounds of the invention have a range of in vitro cell potency activities from about 1 nM to about 10 μM.

### Image Acquirement and Processing:

Assay plates were read on the BD Pathway™ 855 Bioimager equipped with a 488/10 nm EGFP excitation filter, a 380/10 nm DAPI excitation filter, a 515LP nm EGFP emission filter and a 435LP nm DAPI emission filter. Images were acquired in the DAPI and GFP channels of each well using 10× dry objective. The plates were exposed 0.066 ms (Gain 31) to acquire DAPI images and 0.55 ms (Gain 30) for GFP images.

### Data Analysis:

The BD Pathway Bioimager outputs its data in standard text files. Data were imported into the data analysis software BD Image Data Explorer. The nuclear/cytoplasmic (Nuc/Cyt) ratios of fluorescence intensity were determined by dividing the fluorescence intensity of the nucleus by the cytoplasmic. A threshold ratio of greater than 1.8 was employed to define nuclear accumulation of fluorescent signal for each cell. Based on this procedure we calculated the percentage of cells per well displaying nuclear translocation or inhibition of nuclear export. Compounds that induced a nuclear accumulation of the fluorescent signal greater than 60% of that obtained from wells treated with 4 nM LMB were considered as hits. In order to estimate the quality of the HCS assay, the Z′ factor was calculated by the equation: Z′=1−[(3×std. dev. of positive controls)+(3×std. dev. of negative controls)/(mean of positive controls)−(mean of negative controls)].

### PI3K Signalling

### AKT Phosphorylation Inhibition.Western Blot Analysis:

Subconfluent cells were incubated under different conditions and washed twice with TBS prior to lysis. Lysis buffer was added containing 50 mM Tris HCl, 150 mM NaCl, 1% NP-40, 2 mM Na₃VO₄, 100 mM NaF, 20 mM Na₄P₂O₇ and protease inhibitor cocktail (Roche Molecular Biochemicals). The proteins were resolved on 10% SDS-PAGE and transferred to nitrocellulose membrane (Schleicher & Schuell, Dassel, Germany). The membranes were incubated overnight at 4° C. with antibodies specific for Akt, phospho—Ser-473-Akt (Cell Signaling Technology) and α-tubulin (Sigma), they were washed and then incubated with IRDye800 conjugated anti-mouse and Alexa Fluor 680 goat anti-rabbit IgG secondary antibodies. The bands were visualized using an Odyssey infrared imaging system (Li-Cor Biosciences). Compounds of the invention have a range of in vitro cell potency activities from about 1 nM to about 10 μM.

### Cytotoxicity Assessment

The compounds were tested on 96-well trays. Cells growing in a flask were harvested just before they became confluent, counted using a haemocytometer and diluted down with media adjusting the concentration to the required number of cells per 0.2 ml (volume for each well). Cells were then seeded in 96-well trays at a density between 1000 and 4000 cells/well, depending of the cell size. Cells were left to plate down and grow for 24 hours before adding the drugs. Drugs were weighed out and diluted with DMSO to get them into solution to a concentration of 10 mM. From here a “mother plate” with serial dilutions was prepared at 200× the final concentration in the culture. The final concentration of DMSO in the tissue culture media should not exceed 0.5%. The appropriate volume of the compound solution (usually 2 microlitres) was added automatically (Beckman FX 96 tip) to media to make it up to the final concentration for each drug. The medium was removed from the cells and replaced with 0.2 ml of medium dosed with drug. Each concentration was assayed in triplicate. Two sets of control wells were left on each plate, containing either medium without drug or medium with the same concentration of DMSO. A third control set was obtained with the cells untreated just before adding the drugs (seeding control, number of cells starting the culture). Cells were exposed to the drugs for 72 hours and then processed for MTT colorimetric read-out. Compounds of the invention have a range of in vitro cell potency activities from about 1 nM to about 10 μM.

mTOR Assay

Mammalian target of rapamycin (mTOR) was assayed by monitoring phosphorylation of GFP-4EBP using a homogeneous time-resolved fluorescence resonante energy transfer format and assay reagents from Invitrogen. In the presence of 10 μM ATP, 50 mM Hepes (pH 7.5), 0.01% (v/v) Polysorbate 20, 10 mM MnCl₂, 1 mM EGTA, and 2.5 mM DTT, the mTOR-mediated phosphorylation of 200 nM GFP-4E-BP1 was measured under initial rate conditions. After incubation at room temperature for 60 min, the reaction was terminated by addition of 10 mM EDTA, and phosphorylated GFP-4E-BP1 was detected with 2 nM Tb-anti-p4E-BP1 antibody before reading on a Perkin-Elmer Wallac 1420 Fluorescence Reader (exc 340; em 490/520).

Where compound names are given herein, they are typically generated with ChemDraw.

The invention is illustrated by way of the following examples, in which the following abbreviations (or chemical symbols) may be employed: “dba” dibenzylidene acetone; “DCM” dichloromethane; “MeOH” methanol; “EtOH” ethanol; “THF” tetrahydrofuran; “DMF” dimethylformamide; “CHCl₃” chloroform; “DME” dimethoxyethane; “Et₂O” diethyl ether; “Hex” hexane; “EtOAc” ethyl acetate; “Pd(PPh₃)₄” tetrakis(triphenylphosphine)palladium; “KOAc” potassium acetate; “DIPEA” diisopropylethylamine; “Pd(PPh₃)₄” tetrakis(triphenylphosphine)-palladium; “Pd(dppf)Cl₂.DCM” 1,1′-bis(diphenylphosphino)ferrocenepalladium(II) dichloride, dichloromethane; “min.” minutes; and “h.” hours.

### EXAMPLES AND EXPERIMENTAL

### Preparation of Final Product 1-01

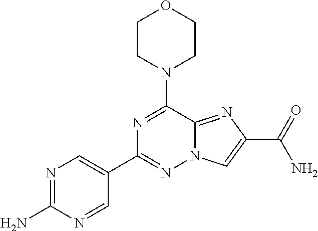 [SMILES: NC(=O)c1cn2nc(-c3cnc(N)nc3)nc(N3CCOCC3)c2n1]

To a solution of Intermediate I-01 (80 mg, 0.272 mmol, 1.0 eq) in DME (2.0 mL) were added 2-aminopyrimidine-5-boronic acid, pinacol ester (120 mg, 0.544 mmol, 2.0 eq), Pd(dppf)Cl₂ (112 mg, 0.136 mmol, 0.5 eq) and Cs₂CO₃ (177 mg, 0.544 mmol, 2.0 eq). The reaction mixture was heated at 130° C. for 18 h. More reagents 2-aminopyrimidine-5-boronic acid, pinacol ester (2.0 eq), Pd(dppf)Cl₂ (0.5 eq) and Cs₂CO₃ (2.0 eq) were added and the reaction mixture was heated at 130° C. for 8 h. On cooling, the mixture was filtered off and the filtrate was evaporated. The residue was dissolved in DCM (100 mL), washed with water (2×50 mL), dried with MgSO₄, filtered and evaporated. The residue was purified by column chromatography (60% EtOAc in cyclohexane and MeOH) and by HPLC-preparative to afford the desired product (2.5 mg, 2.5%).

### Preparation of Intermediate I-01

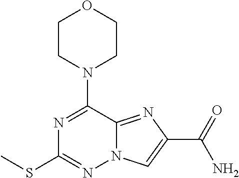 [SMILES: CSc1nc(N2CCOCC2)c2nc(C(N)=O)cn2n1]

A mixture of Intermediate I-02 (80 mg, 0.247 mmol, 1.0 eq) in 7N NH₃ in MeOH (8 mL) was heated in a sealed tube at 95° C. for 4 days. On cooling, the mixture was evaporated and the residue was triturated from MeOH, filtered and washed with DCM. The filtrate was evaporated to afford the desired product (26 mg, 36%).

¹H-NMR (300 MHz, CDCl₃) δ 8.19 (s, 1H), 6.92 (s, 1H), 5.59 (s, 1H), 4.81 (m, 2H), 4.05 (m, 2H), 3.85 (m, 4H), 2.48 (s, 3H).

### Preparation of Intermediate I-02

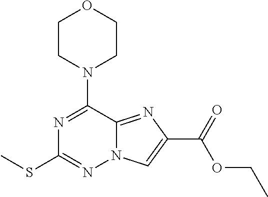 [SMILES: CCOC(=O)c1cn2nc(SC)nc(N3CCOCC3)c2n1]

To a solution of Intermediate I-03 (0.1 g, 0.531 mmol, 1.0 eq) in dry Toluene (10 mL) ethyl bromopyruvate (0.333 mL, 2.65 mmol, 5.0 eq) and p-toluensulfonic acid (16 mg, 0.085 mmol, 0.16 eq) were added. The reaction mixture was heated at 110° C. for 8 h and stirred at rt for 18 h. The mixture was evaporated, the residue was dissolved in DCM (50 mL) and washed with water (2×50 mL). The organic layer was dried (MgSO₄), filtered and evaporated.

The residue was dissolved in MeCN (10 mL) and morpholine (0.464 mL, 0.462 mmol, 10 eq) was added. The reaction mixture was heated at 85° C. for 3 h. On cooling, the mixture was evaporated and the residue was purified by automated column chromatography (Biotage, 20% EtOAc in cyclohexane). The product obtained was triturated from EtOH and filtered to afford the desired product (82 mg, 48%).

¹H NMR (300 MHz, CDCl₃) δ 8.15 (s, 1H), 4.89 (m, 2H), 4.41 (q, J=7.1 Hz, 2H), 4.04 (m, 2H), 3.84 (s, 4H), 2.49 (s, 3H), 1.37 (t, J=7.2 Hz, 3H).

### Preparation of Intermediate I-03

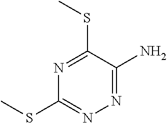

To a solution of Intermediate I-04 (1.496 g, 9.3 mmol, 1 eq) in DCM (70 mL) DIPEA (9.76 mL, 56.0 mmol, 6 eq) and MeI (1.28 mL, 20.5 mmol, 2.2 eq) were added. The reaction mixture was heated at 25° C. for 24 h. The mixture was evaporated and the residue was purified by automated column chromatography (Biotage, EtOAc:cyclohexane, 40:60 to 70:30) to afford the desired product (0.960 g, 55%) as a yellow solid.

¹H NMR (300 MHz, CDCl₃) δ 4.73 (s, 2H), 2.64 (m, 3H), 2.63 (s, 3H).

### Preparation of Intermediate I-04

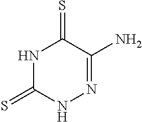

To a solution of Intermediate I-05 (1.328 g, 10.37 mmol, 1.0 eq) and elemental sulfur (91 mg, 2.6 mmol, 0.25 eq) in dry pyridine (90 mL) was added phosphorus pentasulfide (4.61 g, 20.7 mmol, 2.0 eq). The reaction mixture was refluxed for 5 h and allowed to cool upon standing at 25° C. for 18 h. The reddish-brown supernatant liquid was decanted, additional pyridine was added to wash the solid and it was decanted again. Pyridine was removed in vacuo and the resulting residue was covered with water (90 mL), boiled for 30 min and allowed to stand at 4° C. for 6 h. The suspension was filtered and the solid was washed with water and dried in vacuo for 18 h to give a pale brown solid (1.496 g, 90%).

### Preparation of Intermediate I-05

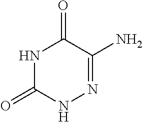

A mixture of Intermediate I-06 (2.5 g, 13.02 mmol, 1 eq), copper (50 mg, 0.781 mmol, 0.06 eq) and liquid ammonia (˜150 mL) were heated in a Parr reactor at 120° C. for 3 days. On cooling, excess of ammonia was vented off and the residual solid was dissolved in MeOH/water (200 mL, 1:1). MeOH was removed in vacuo and the water solution was acidified to pH 4 with HCl. The resulting suspension was filtered off to afford the desired product (1.328 g, 80%) as a white solid.

¹H NMR (300 MHz, DMSO) δ 11.68 (s, 1H), 10.87 (s, 1H), 5.94 (s, 2H).

### Preparation of Intermediate I-06

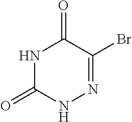

A mixture of 6-Azauracil (5.0 g, 44.2 mmol, 1.0 eq), bromine (5.0 mL, 97 mmol, 2.2 eq) and water (70 mL) was stirred at 25° C. for 27 h. The colourless crystalline precipitate was collected by filtration to give the desired product (4.6 g, 55%).

### Preparation of Final Product 1-02

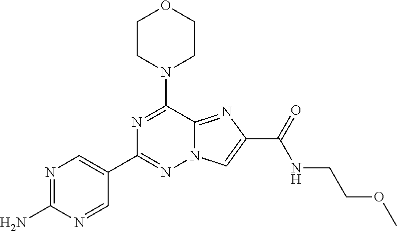 [SMILES: COCCNC(=O)c1cn2nc(-c3cnc(N)nc3)nc(N3CCOCC3)c2n1]

To a solution of Intermediate I-07 (50 mg, 0.142 mmol, 1 eq) in DME (5 mL) were added 2-aminopyrimidine-5-boronic acid, pinacol ester (63 mg, 0.284 mmol, 2 eq), Pd(dppf)Cl₂ (123 mg, 0.149 mmol, 1.05 eq) and Cs₂CO₃ (92 mg, 0.284 mmol, 2 eq). The reaction mixture was heated at 130° C. for 20 h. On cooling, the mixture was filtered off and the filtrate was evaporated. The residue was purified by automated column chromatography (Biotage, 7N NH₃ in MeOH:DCM, 0:100 to 5:95) to afford the desired product (15 mg, 9%) as a white solid.

### Preparation of Intermediate I-07

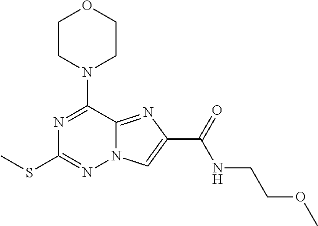

To a solution of 2-methoxyethylamine (0.4 mL, 4.64 mmol, 3 eq) in dry DCM (20 mL) was slowly added a 2 M solution of trimethylaluminium in hexane (2.32 mL) at it under Argon. The mixture was stirred for 30 min and added to a solution of Intermediate I-02 (0.5 g, 1.54 mmol, 1.0 eq) in DCM (20 mL). The reaction mixture was heated at 50° C. overnight. Additional amounts of trimethylaluminium (2.0 eq) and 2-methoxyethylamine (2.0 eq) in DCM (20 mL) were added and the reaction mixture was heated for 8 h. The mixture was quenched with 0.2 M HCl (20 mL) and extracted with DCM (2×100 mL). The combined organic layers were dried (MgSO₄), filtered and concentrated. The residue was triturated from MeOH to afford the desired product (0.385 g, 71%) as a white solid.

### Preparation of Final Product 1-03

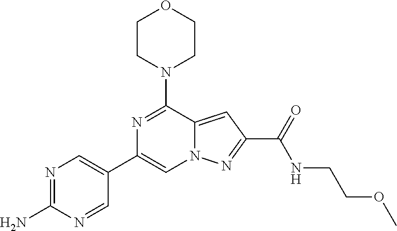 [SMILES: COCCNC(=O)c1cc2c(N3CCOCC3)nc(-c3cnc(N)nc3)cn2n1]

To a solution of Intermediate I-08 (80 mg, 0.13 mmol) in 1,2-DCE (4 mL) at 0° C. trifluoroacetic acid (2 mL) and 98% H₂SO₄ (4 drops) were added. The reaction mixture was stirred at rt for 6 h and stored at −24° C. for 18 h. The mixture was evaporated and the residue was dissolved in water (2 mL) and cooled to 0° C. Aqueous NH₄OH was added up to pH˜8 and the resulting yellow solid was filtered, washed and dried. The solid was purified by column chromatography (EtOAc:MeOH, 99:1) and the product obtained was recrystallized from EtOAc/hexane to give the desired product (32 mg, 64%) as a white solid.

### Preparation of Intermediate I-08

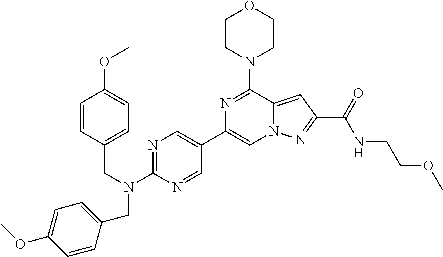

A 2M solution of trimethylaluminium in hexanes (0.33 mL, 0.66 mmol) was added dropwise to a solution of 2-methoxyethylamine (0.055 mL, 0.65 mmol) in dry DCM (5 mL) at rt. The mixture was stirred for 4 h at rt and then added dropwise to a solution of Intermediate I-09 (100 mg, 0.16 mmol) in dry DCM (5 mL). The reaction mixture was refluxed for 18 h. On cooling, the mixture was quenched with 0.5 N HCl (5 mL) and extracted with DCM (4×50 mL). The combined organic fractions were dried (MgSO₄), filtered and evaporated. The residue was purified by column chromatography (EtOAc) to give the desired product (82 mg, 79%) as a white solid.

¹H NMR (300 MHz, CDCl₃) d 8.92 (s, 2H), 8.14 (s, 1H), 7.41 (t, J=5.5 Hz, 1H), 7.26 (s, 1H), 7.21 (d, J=8.6 Hz, 4H), 6.86 (d, J=8.6 Hz, 4H), 4.85 (s, 4H), 3.89 (m, 8H), 3.80 (s, 6H), 3.70 (dd, J=10.4, 5.2 Hz, 2H), 3.60 (t, J=4.9 Hz, 2H), 3.42 (s, 3H).

### Preparation of Intermediate I-09

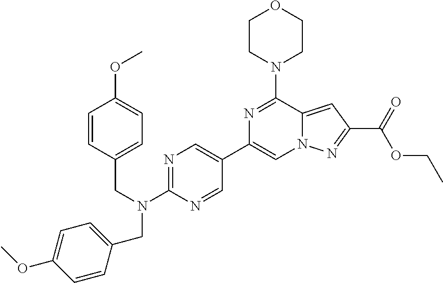 [SMILES: CCOC(=O)c1cc2c(N3CCOCC3)nc(-c3cnc(N(Cc4ccc(OC)cc4)Cc4ccc(OC)cc4)nc3)cn2n1]

A mixture of Intermediate I-10 (1.89 g, 1.19 mmol), morpholine (2.01 mL, 23.8 mmol) and triethylamine (3.36 mL, 23.8 mmol) in dioxane (50 mL) was stirred at rt for 3 days. The mixture was evaporated and the residue was purified by column chromatography (EtOAc:cyclohexane, 10:90) to give the desired product (444 mg, 22%) as a white solid.

¹H NMR (300 MHz, CDCl₃) δ 8.98 (s, 2H), 8.31 (s, 1H), 7.30 (s, 1H), 7.23 (d, J=8.7 Hz, 4H), 6.88 (d, J=8.7 Hz, 4H), 4.92 (s, 4H), 4.51 (q, J=7.0 Hz, 4H), 3.91 (m, 8H), 3.82 (s, 6H), 1.47 (d, J=7.1 Hz, 3H).

### Preparation of Intermediate I-10

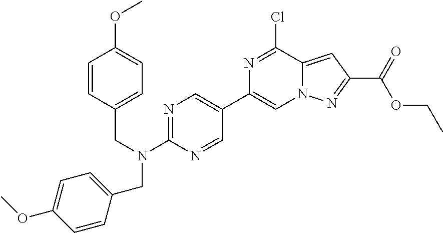 [SMILES: CCOC(=O)c1cc2c(Cl)nc(-c3cnc(N(Cc4ccc(OC)cc4)Cc4ccc(OC)cc4)nc3)cn2n1]

A mixture of Intermediate I-11 (650 mg, 1.19 mmol) and N,N-dimethylaniline (0.5 mL) in POCl₃ (6 mL) was stirred at 80° C. for 21 h. On cooling, more N,N-dimethylaniline (0.5 mL) and POCl₃ (6 mL) were added and the reaction mixture was heated at 90° C. for 24 h. POCl₃ was removed in vacuo, the residue was taken up in DCM (200 mL) and the mixture was poured onto ice. After stirring for 15 minutes, NaHCO₃ was added portionwise up to pH˜8 and the organic layer was separated, washed with water (25 mL), dried (Na₂SO₄), filtered and evaporated to give the desired product (1.89 g) as a blue oil. It was used in the next reaction step without further purification.

### Preparation of Intermediate I-11

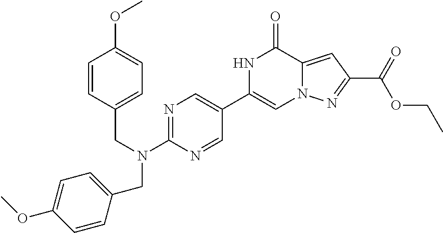 [SMILES: CCOC(=O)c1cc2c(=O)[nH]c(-c3cnc(N(Cc4ccc(OC)cc4)Cc4ccc(OC)cc4)nc3)cn2n1]

A mixture of Intermediate I-12 (220 mg, 0.37 mmol) and NH₄OAc (383 mg, 4.96 mmol) in EtOH (5 mL) was heated under microwave irradiation for 1 h at 150° C. The mixture was cooled to 0° C. and the resulting solid was filtered off, washed with water/EtOH (1:1) and dried to give the desired product (190 mg, 94%) as a white solid.

¹H NMR (300 MHz, DMSO) δ 8.74 (s, 2H), 8.19 (s, 1H), 7.41 (s, 1H), 7.19 (d, J=8.7 Hz, 4H), 6.89 (d, J=8.7 Hz, 4H), 4.78 (s, 4H), 4.34 (q, J=7.1 Hz, 2H), 3.73 (s, 6H), 1.33 (t, J=7.1 Hz, 3H).

### Preparation of Intermediate I-12

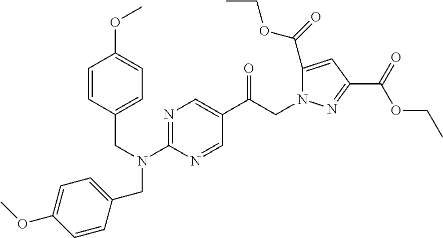 [SMILES: CCOC(=O)c1cc(C(=O)OCC)n(CC(=O)c2cnc(N(Cc3ccc(OC)cc3)Cc3ccc(OC)cc3)nc2)n1]

A mixture of Intermediate I-13 (1.21 g, 2.65 mmol), diethyl 3,5-pyrazoledicarboxylate (539 mg, 2.54 mmol) and K₂CO₃ (440 mg, 3.18 mmol) in acetone (20 mL) was stirred overnight at rt. Water (50 mL) was added and the mixture was extracted with DCM (4×150 mL). The combined organic layers were washed with brine (50 mL), dried (MgSO₄), filtered and evaporated. The residue was purified by column chromatography (EtOAc:cyclohexane, 30:70) and the product obtained was triturated from Et₂O to give the desired product (898 mg, 66%) as a white solid.

¹H NMR (300 MHz, DMSO) δ 9.02 (s, 2H), 7.34 (s, 1H), 7.22 (d, J=8.6 Hz, 4H), 6.89 (d, J=8.7 Hz, 4H), 6.14 (s, 2H), 4.83 (s, 4H), 4.31 (q, J=7.1 Hz, 2H), 4.23 (q, J=7.1 Hz, 2H), 3.73 (s, 6H), 1.31 (t, J=7.1 Hz, 3H), 1.22 (t, J=7.1 Hz, 3H).

### Preparation of Intermediate I-13

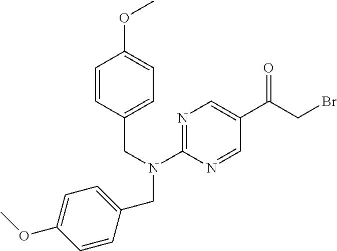 [SMILES: COc1ccc(CN(Cc2ccc(OC)cc2)c2ncc(C(=O)CBr)cn2)cc1]

To a mixture of Intermediate I-14 (879 mg, 2.33 mmol) and TEA (0.96 mL, 7 mmol) in THF (50 mL) at 0° C. was added dropwise trimethylsilyl trifluoromethanesulfonate (1.27 mL, 7 mmol). The reaction mixture was stirred for 2 h at 0° C. and NBS (626 mg, 3.45 mmol) was added portionwise. The mixture was stirred for 1 h at 0° C., diluted with water (30 mL) and extracted with EtOAc (4×150 mL). The combined organic layers were washed with saturated aqueous NaHCO₃, dried (MgSO₄), filtered and evaporated to give the desired product (1.06 g, 100%) as a brown oil.

¹H NMR (300 MHz, DMSO) δ 8.95 (s, 2H), 7.15 (d, J=8.7 Hz, 4H), 6.87 (d, J=8.7 Hz, 4H), 4.79 (s, 4H), 4.00 (q, J=7.1 Hz, 2H), 3.71 (s, 6H), 2.53 (s, 2H), 1.16 (t, J=7.1 Hz, 3H).

### Preparation of Intermediate I-14

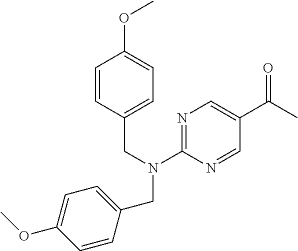 [SMILES: COc1ccc(CN(Cc2ccc(OC)cc2)c2ncc(C(C)=O)cn2)cc1]

A mixture of Intermediate I-15 (1.81 g, 4.37 mmol), tributyl(1-ethoxyvinyl)tin (1.42 mL, 4.24 mmol) and dichlorobis(triphenylphosphine)palladium(II) (0.15 g, 0.21 mmol) in dry DMF (15 mL) was heated at 100° C. for 22 h. On cooling, the reaction mixture was diluted with Et₂O (300 mL) and treated with aqueous 15% KF solution (100 mL). The mixture was vigorously stirred for 1 h, the organic layer was separated and washed with saturated NaHCO₃ (50 mL), brine (50 mL), dried (MgSO₄), filtered and evaporated. The residue was purified by column chromatography (EtOAc:cyclohexane, 10:90) to give the desired product (997 mg, 61%).

¹H NMR (300 MHz, CDCl₃) δ 8.92 (s, 2H), 7.19 (d, J=8.6 Hz, 4H), 6.87 (d, J=8.7 Hz, 4H), 4.85 (s, 4H), 3.78 (s, 6H).

### Preparation of Intermediate I-15

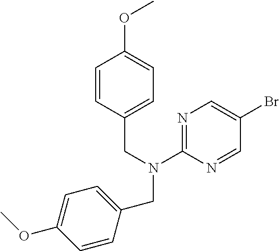 [SMILES: COc1ccc(CN(Cc2ccc(OC)cc2)c2ncc(Br)cn2)cc1]

A mixture of 2-chloro-5-bromopyrimidine (1.0 g, 5.16 mmol), Intermediate I-16 (1.59 g, 5.418 mmol) and DIPEA (2.68 mL, 15.48 mmol) in dry dioxane (10 mL) was heated under microwave irradiation for 1 h at 160° C. On cooling, the mixture was diluted with EtOAc (200 mL), washed with saturated aqueous NaHCO₃ (50 mL), brine (50 mL), dried (MgSO₄), filtered and evaporated. The residue was purified by column chromatography (EtOAc:cyclohexane, 5:95 to 10:90) to give the desired product (1.81 g, 85%) as a white solid.

¹H NMR (300 MHz, DMSO) δ 8.49 (s, 2H), 7.15 (d, J=8.5 Hz, 4H), 6.86 (d, J=8.6 Hz, 4H), 4.68 (s, 4H), 3.72 (s, 6H).

### Preparation of Intermediate I-16

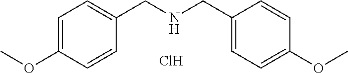

A mixture of p-anisaldehyde (1.8 mL, 14.8 mmol) and 4-methoxybenzylamine (1.9 mL, 14.8 mmol) in EtOH (50 mL) was refluxed for 4 h. On cooling to 0° C., NaBH₄ (562 mg, 14.8 mmol) was added and the reaction mixture was stirred for 18 h at rt. On cooling to 0° C., water (50 mL) and DCM (250 mL) were added. The organic phase was separated and the aqueous layer was extracted with DCM (2×250 mL). The combined organic layers were dried (MgSO₄), filtered and evaporated. The residue was dissolved in Et₂O (100 mL) and the mixture was cooled to 0° C. 4N HCl in dioxane (−10 mL) was added dropwise and the mixture was stirred for 2 h at 0° C. The resulting white solid was filtered off, washed with Et₂O/EtOAc to give the desired product (3.99 g, 91%) as a white solid.

¹H NMR (300 MHz, DMSO) δ 9.40 (s, 2H), 7.45 (d, J=8.7 Hz, 4H), 6.98 (d, J=8.7 Hz, 4H), 4.03 (s, 4H), 3.77 (s, 6H).

### Preparation of Final Product 1-04

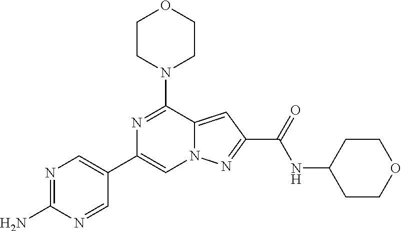 [SMILES: Nc1ncc(-c2cn3nc(C(=O)NC4CCOCC4)cc3c(N3CCOCC3)n2)cn1]

A mixture of Intermediate I-17 (98 mg, 0.29 mmol), HOBt hydrate (116 mg, 0.858 mmol), HBTU (278 mg, 0.74 mmol) and TEA (0.18 mL) in DMF (4 mL) was stirred for 2 h at rt. Then, a mixture, previously stirred for 1 h at rt, of 4-aminotetrahydropyrane (136 mg, 1 mmol) and TEA (0.13 mL) in DMF (1 mL) was added and the reaction mixture was stirred for 72 h at rt. The mixture was evaporated and the residue was purified by column chromatography (reverse phase, water/MeCM mixtures) to give the desired product (16 mg, 13%) as a white solid.

### Preparation of Intermediate I-17

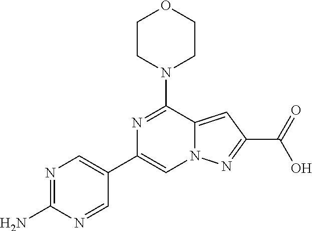 [SMILES: Nc1ncc(-c2cn3nc(C(=O)O)cc3c(N3CCOCC3)n2)cn1]

A mixture of Intermediate I-18 (100 mg, 0.27 mmol), 4N KOH (3 mL) and EtOH (3 mL) was stirred for 4 h at rt. The mixture was evaporated, the residue was cooled to 0° C. and AcOH was added dropwise up to pH˜5. The resulting brown solid was filtered off, washed with cyclohexane and water and dried to give the desired product (95 mg, 100% yield). It was used in the next reaction step without further purification.

¹H NMR (300 MHz, DMSO) d 8.88 (s, 2H), 8.68 (s, 1H), 7.28 (s, 1H), 7.03 (s, 1H), 6.86 (s, 2H), 3.75 (m, 8H).

### Preparation of Intermediate I-18

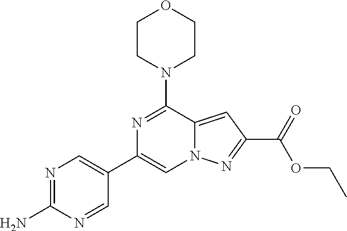 [SMILES: CCOC(=O)c1cc2c(N3CCOCC3)nc(-c3cnc(N)nc3)cn2n1]

To a solution of Intermediate I-09 (136 mg, 0.22 mmol) in 1,2-DCE (5 mL) at 0° C. were added trifluoroacetic acid (4 mL) and 98% H₂SO₄ (4 drops). The reaction mixture was stirred for 4 h at rt. The mixture was evaporated and the residue was cooled to 0° C. and dissolved in water (4 mL). Aqueous NH₄OH was added up to pH˜8 and the resulting white solid was filtered, washed and dried to give the desired product (81 mg, 100%) as a brown solid.

### Preparation of Final Product 1-05

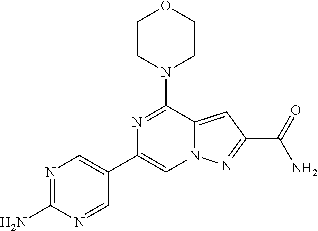 [SMILES: NC(=O)c1cc2c(N3CCOCC3)nc(-c3cnc(N)nc3)cn2n1]

Intermediate I-09 (140 mg, 0.229 mmol) was suspended in a 7N NH₃ solution in MeOH (5 mL) and the resulting solution was heated in a sealed tube at 100° C. for 48 h. On cooling to room temperature, the solvents were removed in vacuo to give the corresponding amide (119 mg, 89%) that, without further purification, was deprotected following a similar procedure described for the synthesis of final product 1-03, to afford 6-(2-amino-pyrimidin-5-yl)-4-morpholin-4-yl-pyrazolo[1,5-a]pyrazine-2-carboxylic acid amide as a yellowish solid (14.0 mg) after purification by column chromatography (DCM/7 N MeOH 3-30% as eluent).

### Preparation of Final Product 1-06

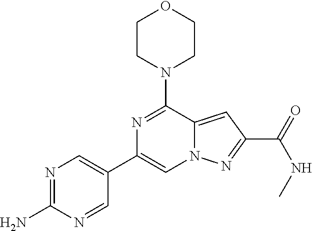 [SMILES: CNC(=O)c1cc2c(N3CCOCC3)nc(-c3cnc(N)nc3)cn2n1]

Intermediate I-09 (70 mg, 0.115 mmol) was suspended in a 2M MeNH₂ solution in MeOH (5 mL) and the resulting solution was heated in a sealed tube at 100° C. for 48 h. On cooling to room temperature, the solvents were removed in vacuo to give, after purification by column chromatography using EtOAc as eluent the corresponding methylamide (55.8 mg, 81%) that was deprotected following a similar procedure described for the synthesis of final product 1-03, to afford 6-(2-amino-pyrimidin-5-yl)-4-morpholin-4-yl-pyrazolo[1,5-a]pyrazine-2-carboxylic acid methylamide, final product 1-06, as a yellowish solid (14.0 mg) after purification by column chromatography (DCM/MeOH 5-10% as eluent).

### Preparation of Final Product 1-07

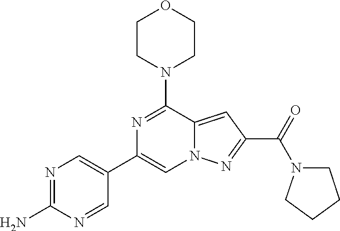 [SMILES: Nc1ncc(-c2cn3nc(C(=O)N4CCCC4)cc3c(N3CCOCC3)n2)cn1]

A 2.0 M solution of Me₃Al in hexanes (0.24 mL, 0.46 mmol) was added dropwise to a solution of pyrrolidine (33 mg, 0.46 mmol) in dry DCM (4 mL) at 25° C. After stirring for 4 h at this temperature, the resulting solution was added dropwise to a solution of intermediate I-09 (70 mg, 0.115 mmol) in dry DCM (5 mL) at 25° C. and the reaction mixture was refluxed for 18 h. On cooling to 0° C., the mixture was quenched with 0.5 N HCl and extracted with DCM (4×). The combined organic fractions were dried and the solvents removed in vacuo to give a residue that was purified by column chromatography (EtOAc as eluent) to give the corresponding pyrrolidin amide (52 mg, 71%) that was deprotected, following a similar procedure described for the synthesis of final product 1-03, to afford final product 1-07, [6-(2-amino-pyrimidin-5-yl)-4-morpholin-4-yl-pyrazolo[1,5-a]pyrazin-2-yl]-pyrrolidin-1-yl-methanone as a yellowish solid (24.0 mg) after purification by column chromatography (DCM/MeOH 5-10% as eluent).

### Preparation of Final Product 1-08

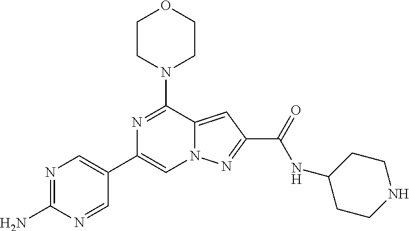 [SMILES: Nc1ncc(-c2cn3nc(C(=O)NC4CCNCC4)cc3c(N3CCOCC3)n2)cn1]

A 2.0 M solution of Me₃Al in hexanes (0.50 mL, 0.92 mmol) was added dropwise to a solution of Piperidin-4-ylamine (92 mg, 0.92 mmol) in dry DCM (8 mL) at 25° C. After stirring for 4 h at this temperature, the resulting solution was added dropwise to a solution of intermediate I-09 (140 mg, 0.230 mmol) in dry DCM (10 mL) at 25° C. and the reaction mixture was refluxed for 18 h. On cooling to 0° C., the mixture was quenched with 0.5 N HCl and extracted with DCM (4×). The combined organic fractions were dried and the solvents removed in vacuo to give a residue that was purified by column chromatography (DCM/MeOH 1-10% as eluent) to give the corresponding amide (68.3 mg, 45%) that was deprotected, following a similar procedure described for the synthesis of final product 1-03, to afford final product 1-08, 6-(2-amino-pyrimidin-5-yl)-4-morpholin-4-yl-pyrazolo[1,5-a]pyrazine-2-carboxylic acid piperidin-4-ylamide as a yellowish solid (17.0 mg) after purification by column chromatography (DCM/7N NH₃ in MeOH 5-10% as eluent).

### Preparation of Final Product 1-09

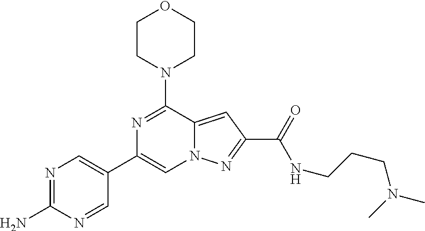 [SMILES: CN(C)CCCNC(=O)c1cc2c(N3CCOCC3)nc(-c3cnc(N)nc3)cn2n1]

A 2.0 M solution of Me₃Al in hexanes (0.50 mL, 0.92 mmol) was added dropwise to a solution of N,N-dimethyl-1,3-propanediamine (94.0 mg, 0.92 mmol) in dry DCM (8 mL) at 25° C. After stirring for 4 h at this temperature, the resulting solution was added dropwise to a solution of intermediate I-09 (140 mg, 0.230 mmol) in dry DCM (10 mL) at 25° C. and the reaction mixture was refluxed for 18 h. On cooling to 0° C., the mixture was quenched with 0.5 N HCl and extracted with DCM (4×). The combined organic fractions were dried and the solvents removed in vacuo to give a residue that was purified by column chromatography (DCM/MeOH 1-10% as eluent) to give the corresponding amide (61 mg, 48%) that was deprotected, following a similar procedure described for the synthesis of final product 1-03, to afford final product 1-09 6-(2-amino-pyrimidin-5-yl)-4-morpholin-4-yl-pyrazolo[1,5-a]pyrazine-2-carboxylic acid (3-dimethylamino-propyl)-amide as a yellowish solid (18.0 mg) after purification by column chromatography (DCM/7N NH₃ in MeOH 5-10% as eluent).

### Preparation of Final Product 1-10

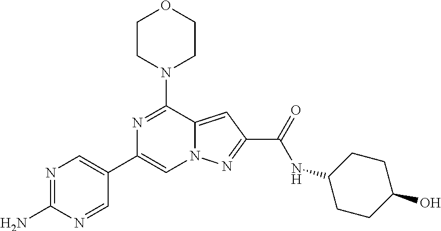

A 2.0 M solution of Me₃Al in hexanes (0.50 mL, 0.92 mmol) was added dropwise to a solution of trans-4-[(tert-Butyldimethylsilanyl)oxy]cyclohexyl]amine, CAS: 690992-74-6, (211 mg, 0.92 mmol) in dry DCM (8 mL) at 25° C. After stirring for 4 h at this temperature, the resulting solution was added dropwise to a solution of intermediate I-09 (140 mg, 0.230 mmol) in dry DCM (10 mL) at 25° C. and the reaction mixture was refluxed for 18 h. On cooling to 0° C., the mixture was quenched with 0.5 N HCl and extracted with DCM (4×). The combined organic fractions were dried and the solvents removed in vacuo to give a residue that, without purification, was deprotected, following a similar procedure described for the synthesis of final product 1-03, to afford final compound 1-11, 6-(2-amino-pyrimidin-5-yl)-4-morpholin-4-yl-pyrazolo[1,5-a]pyrazine-2-carboxylic acid (4-hydroxy-cyclohexyl)-amide as a yellowish solid (10.0 mg) after purification by column chromatography (DCM/7N NH₃ in MeOH 5-10% as eluent).

### General Procedure

The HPLC measurement was performed using a HP 1100 from Agilent Technologies comprising a pump (binary) with degasser, an autosampler, a column oven, a diode-array detector (DAD) and a column as specified in the respective methods below. Flow from the column was split to a MS spectrometer. The MS detector was configured with an electrospray ionization source or API/APCI. Nitrogen was used as the nebulizer gas. The source temperature was maintained at 150° C. Data acquisition was performed with ChemStation LC/MSD quad, software.

### Method 1

Reversed phase HPLC was carried out on a Gemini-NX C18 (100×2.0 mm; 5 um), Solvent A: water with 0.1% formic acid; Solvent B: acetonitrile with 0.1% formic acid. Gradient: 5% of B to 100% of B within 8 min at 50° C., DAD.

### Method 2

Reversed phase HPLC was carried out on a Gemini-NX C18 (100×2.0 mm; 5 um), Solvent A: water with 0.1% formic acid; Solvent B: acetonitrile with 0.1% formic acid. Gradient: 5% of B to 40% of B within 8 min at 50° C., DAD.

TABLE 1
Analytical data and PI3Kα activity - Rₜ means retention time (in minutes), [M + H]⁺
means the protonated mass of the compound, method refers to the method used for (LC)MS.
Biological activity in PI3Kα for certain representative examples is represented in Table 1:

| Cpd. | | | | PI3Kα | |
|---|---|---|---|---|---|
| Nr. | Rₜ | [M + 1]⁺ | Meth. | IC50 | ¹H NMR (300 MHz; δ in ppm, J in Hz) |
| 1-01 | 2.99 | 342.1 | 1 | 19 nM | DMSO δ 8.98 (s, 2H), 8.38 (s, 1H), 7.91 (s, |
| | | | | (<100 nM) | 1H), 7.48 (s, 1H), 7.19 (s, 2H), 4.87 (m, |
| | | | | | 2H), 4.11 (m, 2H), 3.79 (m, 4H). |
| 1-02 | 3.45 | 400.2 | 1 | 3 nM | DMSO δ 8.98 (s, 2H), 8.44 (s, 1H), 8.40 (s, |
| | | | | (<100 nM) | 1H), 7.21 (s, 2H), 4.87 (s, 2H), 4.11 (s, 2H), |
| | | | | | 3.81 (s, 5H), 3.42 (d, J = 28.0, 5H), 3.27 (s, 3H). |
| 1-03 | 3.39 | 399.1 | 1 | 29 nM | DMSO δ 8.89 (s, 2H), 8.65 (s, 1H), 8.29 |
| | | | | (<100 nM) | (m, 1H), 7.45 (s, 1H), 6.91 (s, 2H), 3.81 (m, |
| | | | | | 8H), 3.48 (m, 4H), 3.28 (s, 3H). |
| 1-04 | 3.42 | 425.3 | 1 | 26 nM | DMSO δ 8.87 (s, 2H). 8.62 (s, 1H), 8.30 (d, |
| | | | | (<100 nM) | J = 7.9, 1H), 7.44 (s, 1H), 6.90 (s, 2H), 4.05 |
| | | | | | (m, 1H), 3.84 (m, 10H), 3.39 (m, 2H), 1.68 |
| | | | | | (m, 4H). |
| 1-05 | 4.028 | 340.9 | 2 | 14 nM | DMSO δ 8.89 (s, 2H), 8.63 (s, 1H), 7.76 (s, |
| | | | | | 1H), 7.56 (s, 1H), 7.44 (s, 1H), 6.89 (s, 2H), |
| | | | | | 3.81 (d, J = 5.1 Hz, 8H). |
| 1-06 | 4.650 | 355.0 | 2 | — | DMSO δ 8.83 (s, 2H), 8.54 (s, 1H), 8.32 (d, |
| | | | | | J = 4.5 Hz, 1H), 7.35 (s, 1H), 6.84 (s, 2H), |
| | | | | | 3.75 (d, J = 4.6 Hz, 2H), 2.75 (d, J = 4.6 |
| | | | | | Hz, 3H). |
| 1-07 | 5.814 | 395.0 | 2 | 9 nM | DMSO δ 8.90 (s, 2H), 8.71 (s, 1H), 7.37 (s, |
| | | | | | 1H), 6.88 (s, 2H), 3.88 (t, J = 6.4 Hz, 2H), |
| | | | | | 3.80 (d, J = 4.5 Hz, 8H), 3.54 (t, J = 6.5 Hz, |
| | | | | | 1H), 1.89 (m, 4H). |
| 1-08 | 0.493 | 424.0 | 2 | — | — |
| 1-09 | 0.434 | 426.0 | 2 | 443 nM | DMSO δ 8.95 (s, 2H), 8.71 (t, J = 6.0 Hz, |
| | | | | | 1H), 8.64 (s, 1H), 7.50 (s, 1H), 6.98 (s, 2H), |
| | | | | | 3.87 (d, J = 5.7 Hz, 8H), 3.44 (m, 2H), 3.15 |
| | | | | | (m, 2H), 2.83 (s, 6H), 1.96 (m, 2H). |
| 1-10 | 5.157 | 439.0 | 2 | 16 nM | DMSO δ 8.87 (s, 2H), 8.62 (s, 1H), 8.07 (d, |
| | | | | | J = 8.2 Hz, 1H), 7.42 (s, 1H), 6.90 (s, 2H), |
| | | | | | 4.56 (d, J = 4.3 Hz, 1H), 3.81 (m, 9H), 3.17 |
| | | | | | (d, J = 5.3 Hz, 1H), 1.83 (s, 4H), 1.32 (m, 4H). |

Cellular activity of some representative compounds (AKT phosphorylation; see biological example above) display an inhibition <1 μM.

1. A compound of formula I, 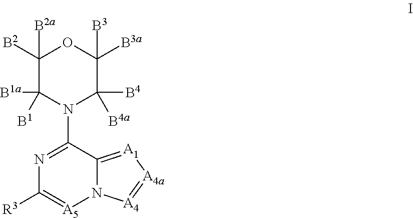
wherein:
A₁ represents N or C(R¹);
A₄ represents N or C(R^{1a});
A₄ₐ represents N or C(R^{1b});
wherein at least one of A₄ and A₄ₐ does not represent N;
A₅ represents N or C(R²);
each B¹, B^{1a}, B², B^{2a}, B³, B^{3a}, B⁴ and B^{4a} independently represent hydrogen or a substituent selected from halo, —C(═Y)—R^{10a}, —C(═Y)—OR^{10a}, —C(═Y)N(R^{10a})R^{11a}, —S(O)₂N(R^{10a})R^{11a}, C₁₋₁₂ alkyl, heterocycloalkyl (which latter two groups are optionally substituted by one or more substituents selected from ═O and E¹), aryl and/or heteroaryl (which latter two groups are optionally substituted by one or more substituents selected from E²); or
any two B¹, B², B^{2a}, B³, B^{3a}, B⁴ and B^{4a} substituents that are attached to the same carbon atom may together form a ═O group;
or, any two B¹, B^{1a}, B², B^{2a}, B³, B^{3a}, B⁴ and B^{4a} substituents may be linked together to form a further 3- to 12-membered ring, optionally containing (in addition to the atom(s) of the morpholine ring) one or more heteroatom(s), which ring optionally contains one or more double bonds, and which ring is itself optionally substituted by one or more substituents selected from halo, ═O and C₁₋₃ alkyl optionally substituted by one or more fluoro atoms;
R¹ and R² (when present) independently represent hydrogen or a substituent selected from halo, —ON, —OR^{10b}, —N(R^{10b})R^{11b}, —C(O)N(R^{10b})R^{11b}, C₁₋₁₂ alkyl and heterocycloalkyl, which latter two groups are optionally substituted by one or more substituents selected from E³ and ═O;
one of R^{1a} and R^{1b} is present and represents —C(═Y)N(R^{10a})R^{11a} or —C(═Y)—R^{10a} and the other independently represents hydrogen or Q¹;
Q¹ represents:
halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, alkyl, heterocycloalkyl (which latter two groups are optionally substituted by one or more substituents selected from ═O and E⁴), aryl or heteroaryl (which latter two groups are optionally substituted by one or more substituents selected from E^{4a});
R³ represents:
(i) a bicyclic aryl or heteroaryl group (both of which are optionally substituted by one or more substituents selected from E⁵); or
(ii) a fragment of formula IA or IB, 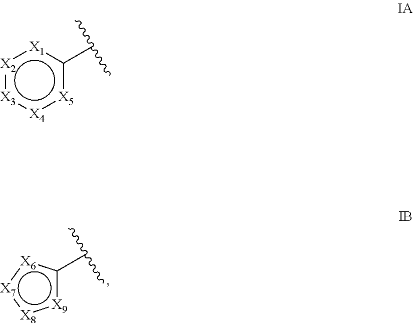
wherein
any one of X₁, X₂, X₃, X₄ and X₅ represents C(R^{2a}), a second one of X₁, X₂, X₃, X₄ and X₅ represents C(R^{2b}), and the remaining three independently represent C(R^{2b}) or N; or
any one, two or three of X₁, X₂, X₃, X₄ and X₅ may represent N and those remaining represent C(H);
X₆, X₇, X₈ and X₉ independently represent C(R^{2b}), N, O or S;
each R^{2b} represents hydrogen, halo, —CN or R^{2a};
each R^{2a} independently represents, on each occasion when used herein, —N(R^{5a})R^{5b}, —N(R^{5c})—C(═Y)—R^{5d}, —N(R^{5e})—C(═Y)—N(R^{5f}), —N(R^{5g})—C(O)—OR^{5h}, —N(R⁵ⁱ)—OR^{5j}, —OR^{5k}, —N(R^{5m})—S(O)₂—R⁵ⁿ or C₁₋₁₂ alkyl optionally substituted by one or more halo atoms;
each R^{5a}, R^{5b}, R^{5c}, R^{5d}, R^{5e}, R^{5f}, R^{5g}, R^{5h}, R⁵ⁱ, R^{5j}, R^{5k}, R^{5m} and R⁵ⁿ independently represent hydrogen, C₁₋₁₂ alkyl (optionally substituted by one or more halo atoms), heterocycloalkyl, aryl or heteroaryl (which latter three groups are optionally substituted by one or more substituents selected from halo and C₁₋₄ alkyl);
each R^{10a}, R^{11a}, R^{10b}, R^{11b} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁶), aryl or heteroaryl (which latter two groups are optionally substituted by one or more substituents selected from E⁷); or
any relevant pair of R^{10a} and R^{11a} or R^{10b} and R^{11b} may be linked together to form a 4- to 20-membered ring, optionally containing one or more heteroatoms, optionally containing one or more unsaturations, and which ring is optionally substituted by one or more substituents selected from ═O, ═S, ═N(R²⁰) and E⁸;
each E¹, E², E³, E⁴, E^{4a}, E⁵, E⁶, E⁷ and E⁸ independently represents, on each occasion when used herein:
(i) Q⁴;
(ii) C₁₋₁₂ alkyl optionally substituted by one or more substituents selected from ═O and Q⁵; or
any two E¹, E², E³, E⁴, E^{4a}, E⁵, E⁶, E⁷ and E⁸ groups may be linked together to form a 3- to 12-membered ring, optionally containing one or more unsaturations, and which ring is optionally substituted by one or more substituents selected from ═O and J¹;
each Q⁴ and Q⁵ independently represent, on each occasion when used herein:
halo, —CN, —NO₂, —N(R²⁰)R²¹, —OR²⁰, —C(═Y)—R²⁰, —C(═Y)—OR²⁰, —C(═Y)N(R²⁰)R²¹, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —Op(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may be linked together to form a 4- to 20-membered ring, optionally containing one or more heteroatoms, optionally containing one or more unsaturations, and which ring is optionally substituted by one or more substituents selected from J⁶ and ═O;
each J¹, J², J³, J⁴, J⁵ and J⁶ independently represents, on each occasion when used herein:
(i) Q⁷;
(ii) C₁₋₆ alkyl or heterocycloalkyl, both of which are optionally substituted by one or more substituents selected from ═O and Q⁸;
each Q⁷ and Q⁸ independently represents, on each occasion when used herein:
halo, —CN, —N(R⁵⁰)R⁵¹, —OR⁵⁶, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may be linked together to form, a 3- to 8-membered ring, optionally containing one or more heteroatoms, optionally containing one or more unsaturations, and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically acceptable ester, amide, solvate or salt thereof.

2. The compound as claimed in claim 1, wherein the requisite A₁, A₄, A₄ₐ and A₅-containing bicyclic core represents any one of the following:

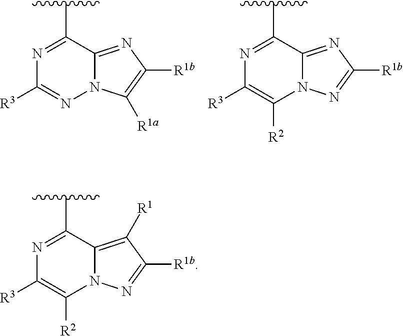

3. The compound as claimed in claim 1, wherein:
either (i) R^{1b} is present and represents —C(═Y)N(R^{10a})R^{11a} or —C(═Y)—R^{10a} and R^{1a} (if present) represents hydrogen or Q¹ as hereinbefore defined; or (ii) R^{1a} is present represents —C(═Y)N(R^{10a})R^{11a} or —C(═Y)—R^{10a} and either R^{1b} is not present or R^{1b} is present and represents hydrogen or Q¹, in which Q¹ represents halo, —CN, —NO₂, —N(R^{10a})R^{11a}, —OR^{10a}, —C(═Y)—R^{10a}, —C(═Y)N(R^{10a})R^{11a}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, aryl or heteroaryl (which latter two groups are optionally substituted by one or more substituents selected from E^{4a}); and/or
R³ represents substituted phenyl, optionally substituted indazolyl, pyrimidinyl, azaindolyl, indolyl or pyridyl.

4. The compound as claimed in claim 1, wherein: R² represents hydrogen, chloro, bromo, iodo or —CN; each R^{10a}, R^{11a}, R^{10b}, R^{11b} and R^{12a} independently represents hydrogen or C₁₋₄ alkyl, which alkyl group may by substituted by one or more substituents selected from ═O and E⁶; or any relevant pair of R^{10a} and R^{11a} and/or R^{10b} and R^{11b}, may be linked together to form a 5- or a 6-membered ring, optionally containing a further heteroatom, and optionally substituted by one or more substituents selected from ═O and E⁸ (which E⁸ substituent may be situated on a nitrogen heteroatom; and/or E⁸ is halo or C₁₋₃ alkyl optionally substituted by one or more fluoro atoms); and/or each E¹, E², E³, E⁴, E^{4a}, E⁵, E⁶, E⁷ and E⁸ independently represent C₁₋₄ alkyl optionally substituted by one or more Q⁵ substituents, or, each of these represent a substituent selected from Q⁴.

5. The compound as claimed in claim 1, wherein: Q⁴ and Q⁵ independently represent halo, OR²⁰, —N(R²⁰)R²¹, —C(═Y)OR²⁰, —C(═Y)N(R²⁰)R²¹, —NR²²S(O)₂R²⁰, heterocycloalkyl, aryl, heteroaryl (which latter three groups are optionally substituted with one or more substitutents selected from J² or J³, as appropriate) and/or C₁₋₆ alkyl optionally substituted by one or more fluoro atoms; each Y represents, on each occasion when used herein, ═S or ═O; each R²⁰, R²¹, R²² and R²³ independently represents hydrogen or C₁₋₄ alkyl optionally substituted by one or more J⁴ substituent(s); or any relevant pair of R²⁰, R²¹ and R²² may may be linked together to form a 5- or a 6-membered ring, optionally containing a further heteroatom, and optionally substituted by one or more substituents selected from ═O and J⁶ (which J⁶ substituent may be situated on a nitrogen heteroatom); R²² represents C₁₋₃ alkyl or hydrogen; each J¹, J², J³, J⁴, J⁵ and J⁶ independently represent a substituent selected from Q⁷, or J¹ to J⁶ represents C₁₋₆ alkyl; each Q⁷ and Q⁸ independently represent —C(═Y)—OR⁵⁰, —C(═Y^{a})—R⁵⁰, —S(O)₂R⁵⁰ or C₁₋₃ alkyl optionally substituted by one or more fluoro atoms; each Y^{a} independently represents ═S or ═O; and/or each R⁵⁰ independently represents C₁₋₄ alkyl.

6. The compound of formula I as defined in claim 1, or a pharmaceutically acceptable ester, amide, solvate or salt thereof, for use as a pharmaceutical.

7. A pharmaceutical formulation comprising a compound of formula I, as defined in claim 1, or a pharmaceutically acceptable ester, amide, solvate or salt thereof, in admixture with a pharmaceutically acceptable adjuvant, diluent or carrier.

8. (canceled)

9. (canceled)

10. (canceled)

11. A method of treatment of a disease in a subject in which inhibition of a PI3-K and/or mTOR is desired and/or required, which method comprises administration of a therapeutically effective amount of a compound of formula I as defined in claim 1, or a pharmaceutically-acceptable ester, amide, solvate or salt thereof, to a patient suffering from, or susceptible to, such a condition.

12. A combination product comprising:
(A) a compound of formula I as defined in claim 1, or a pharmaceutically-acceptable ester, amide, solvate or salt thereof; and
(B) another therapeutic agent that is useful in the treatment of in the treatment of cancer and/or a proliferative disease,
wherein each of components (A) and (B) is formulated in admixture with a pharmaceutically-acceptable adjuvant, diluent or carrier.

13. A process for the preparation of a compound of formula I as defined in claim 1, which process comprises:
(i) reaction of a compound of formula II, 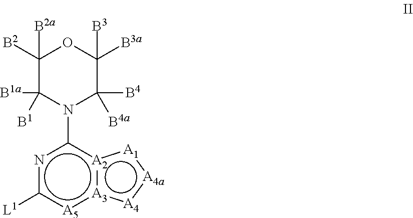
wherein L¹ represents a suitable leaving group, and A₁, A₂, A₃, A₄, A₄ₐ, A₅, B¹, B^{1a}, B², B^{2a}, B³, B^{3a}, B⁴ and B^{4a} are as defined in claim 1, with a compound of formula III,
R³-L² III
wherein L² represents a suitable group;
(ii) reaction of a compound of formula IV, 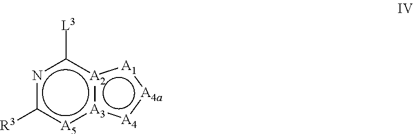
wherein L³ represents a suitable leaving group, and A₁, A₂, A₃, A₄, A₄ₐ, A₅ and R³ as defined in claim 1, with a compound of formula V, 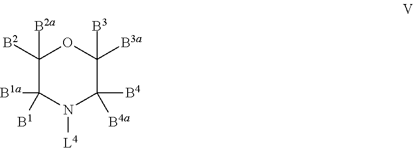
wherein L⁴ may represent hydrogen (so forming an amine group), and B¹, B^{1a}, B², B^{2a}, B³, B^{3b}, B⁴ and B^{4a} are as defined in claim 1, and L¹ is as defined above;
(iii) for compounds of formula I in which R² represents halo, reaction of a corresponding compound of formula I, in which R² represents hydrogen, with a reagent that is a source of halide ions (a halogenating reagent);
(iv) for compounds of formula I in which R² (if present) represents a substituent other that hydrogen, or halo, reaction of a corresponding compound of formula I, in which R² represents halo, with a compound of formula VI,
R^{2aa}-L⁷ VI
wherein R^{2aa} represents R² as described in claim 1 provided that it does not represent hydrogen or halo, and L⁷ represents a suitable leaving group;
(v) for compounds of formula I in which R^{1a} and/or R^{1b} represents —C(O)N(R^{10a})R^{11a}, reaction of a compound of formula VIA, 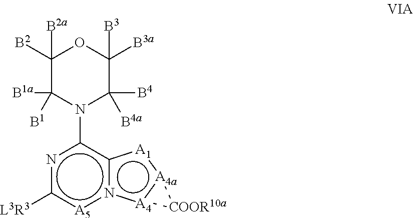
wherein L¹R³ represents either L¹ as defined above or R³ as defined in claim 1, and wherein either A₄ or A₄ₐ represents C(R^{1a}) or C(R^{1b}) respectively, in which either R^{1a} or R^{1b} represents the relevant —COOR^{10a} moiety, and the other one of A₄ or A₄ₐ represents N or C(R^{1a}) or C(R^{1b}) (as appropriate) in which the other R^{1a} or R^{1b} group represents hydrogen or Q¹ as hereinbefore defined, and B¹, B^{1a}, B², B^{2a}, B³, B^{3a}, B⁴, A₁, A₄, A₄, R³ and R^{10a} are as defined in claim 1, with a compound of formula VIB,
HN(R^{10a})R^{11a} VIB
wherein R^{10a} and R^{11a} are as defined in claim 1;
(vi) for compounds of formula I in which R^{1a} and/or R^{1b} is present that represents halo or —C(O)OR^{10a} reaction of corresponding compounds of formula I in which R^{1a} or R^{1b} (as appropriate) represents hydrogen, with a suitable base, followed by reaction in the presence of an electrophile that is a source of halide ions, or CO₂ (to form compounds of formula I in which R^{1a} and/or R^{1b} represent —COOH) or a compound of formula VII,
L⁸-R^{1b1} VII
wherein L⁸ represents a suitable leaving group, such as one hereinbefore defined in respect of L¹ above, and R^{1b1} represents —C(O)OR^{10a};
(vii) for compounds of formula I which contain a —C(OH)(H)—C₁₋₁₁ alkyl group (which alkyl group may be substituted by one or more substituents selected from those defined herein, reaction of a corresponding compound of formula I in which there is a —C(O)H group present, with a compound of formula VIII,
R^{XX}MgX¹ VIII
wherein R^{XX} represents C₁₋₁₁ alkyl optionally substituted by one or more substituents selected from E³ and ═O and X¹ represents halo;
(viii) compounds of formula I in which A₁ and A₄ both represent N, A₅ represents C(R²) and A₄ₐ represents C(R^{1b}) may be prepared by reaction of a compound of formula IX, 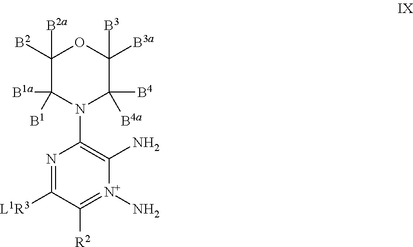
wherein L¹R³ represents either L¹ as defined above or R³ as defined in claim 1, and R², B¹, B^{1a}, B², B^{2a}, B³, B^{3a}, B⁴ and B^{4a} are as defined in claim 1, with a compound of formula X,
H—C(O)—R^{1b} X
wherein R^{1b} represents —C(═Y)N(R^{10a})R^{11a} or —C(═Y)R^{10a};
(ix) compounds of formula I in which A₁ represents N, A₄ represents C(R^{1a}), A₄ₐ represents N and A₅ represents C(R²) may be prepared by reaction of a compound of formula XI, 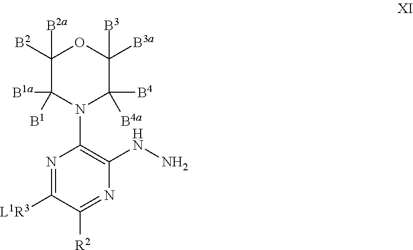
wherein L¹R³, R², B¹, B^{1a}, B², B^{2a}, B³, B^{3a}, B⁴ and B^{4a} are as defined above/in claim 1, with a compound of formula XII,
R^{1a}—C(OC₁₋₆alkyl)₃ XII
or, a compound of formula XIII,
R^{1a}—C(O)OH XIII
or, derivatives of either, wherein R^{1a} represents —C(═Y)N(R^{10a})R^{11a} or —C(═Y)R^{10a} as defined above/in claim 1;
(x) for compounds of formula I that contain an unsubstituted amino group, may be prepared by reaction/deprotection of a corresponding amino protected compound of formula I;
(xi) for compounds of formula I that contain an amino group attached to an aromatic group, reaction of a compound corresponding to a compound of formula but in which there is a halo group in that position by reaction of an amine.

14. A process for the preparation of a pharmaceutical formulation, which process comprises bringing into association a compound of formula I, as defined in claim 1, or a pharmaceutically acceptable ester, amide, solvate or salt thereof with a pharmaceutically-acceptable adjuvant, diluent or carrier.

15. A process for the preparation of a combination product, which process comprises bringing into association a compound of formula I, as defined in claim 1, or a pharmaceutically acceptable ester, amide, solvate or salt thereof with the other therapeutic agent that is useful in the treatment of cancer and/or a proliferative disease, and at least one pharmaceutically-acceptable adjuvant, diluent or carrier.

16. The method according to claim 11, wherein the disease is cancer, an immune disorder, a cardiovascular disease, a viral infection, inflammation, a metabolism/endocrine function disorder, a neurological disorder, an obstructive airways disease, an allergic disease, an inflammatory disease, immunosuppression, a disorder commonly connected with organ transplantation, an AIDS-related disease, benign prostate hyperplasia, familial adenomatosis, polyposis, neuro-fibromatosis, psoriasis, a bone disorder, atherosclerosis, vascular smooth cell proliferation associated with atherosclerosis, pulmonary fibrosis, arthritis glomerulonephritis and post-surgical stenosis, restenosis, stroke, diabetes, hepatomegaly, Alzheimer's disease, cystic fibrosis, a hormone-related disease, an immunodeficiency disorder, a destructive bone disorder, an infectious disease, a condition associated with cell death, thrombin-induced platelet aggregation, chronic myelogenous leukaemia, liver disease, a pathologic immune condition involving T cell activation, or a CNS disorder.
